(19) United States
(12) Patent Application Publication
(10) Pub. No.: US 2007/0128586 A1
(43) Pub. Date: Jun. 7, 2007

(54) Therapeutic, prophylactic and diagnostic agents

(76) Inventors: Kumar Visvanathan, Victoria (AU); Stephen Riordan, New South Wales (AU); Stephen Locarnini, Victoria (AU)

Correspondence Address: The McCallum Law Firm, 132 Kolar Ct, Erle, CO, 80516, US

(21) Appl. No.: 10/550,198

(22) PCT Filed: Mar. 19, 2004
(86) PCT No.: PCT/AU04/00349

§ 371 Date: Nov. 7, 2006

(30) Foreign Application Priority Data
Mar. 21, 2003 (AU) 2003901325

(51) Int. Cl. C12Q 1/70; C12Q 1/68; G01N 33/574; G01N 33/554; A61K 39/395; G01N 33/569; A61K 48/00

(52) U.S. Cl. 435/5; 435/6; 435/7.32; 435/7.23; 424/144.1; 514/44

(57) ABSTRACT

The present invention provides compounds useful in the treatment and prophylaxis of infection in mammals and avian species by pathogenic agents such as, but not limited to, viruses. The present invention further provides compounds useful in the treatment of other disease conditions such as cirrhosis and hepatocellular carcinoma. The present invention further provides methods for diagnosing infection by pathogenic organisms and viruses or other disease conditions and agents useful in diagnostic protocols. The present invention further contemplates methods for monitoring disease states and providing an indication of the susceptibility of a subject for infection by a pathogenic organism or virus or development of other diseased states. In particular, the present invention enables a determination of whether, including a prediction of the level of likelihood that, a subject will respond to therapeutic or prophylactic intervention of an infection or disease condition.

The present application is a U.S. national phase filing under 35 U.S.C. 371 of PCT application No. PCT/AU 2004/00349, filed Mar. 19, 2004, which claim the benefit of Australian patent Application No. 2003901325, filed Mar. 21, 2003 each of which are hereby incorporated by reference in its entirety.

### BACKGROUND OF THE INVENTION

1. Field of the Invention

The present invention provides compounds useful in the treatment and prophylaxis of infection in mammals and avian species by pathogenic agents such as, but not limited to, viruses. The present invention further provides compounds useful in the treatment of other disease conditions such as cirrhosis and hepatocellular carcinoma. The present invention further provides methods for diagnosing infection by pathogenic organisms and viruses or other disease conditions and agents useful in diagnostic protocols. The present invention further contemplates methods for monitoring disease states and providing an indication of the susceptibility of a subject for infection by a pathogenic organism or virus or development of other diseased states. In particular, the present invention enables a determination of whether, including a prediction of the level of likelihood that, a subject will respond to therapeutic or prophylactic intervention of an infection or disease condition.

2. Description of the Prior Art

Bibliographic details of the publications referred to in this specification are also collected at the end of the description.

Reference to any prior art in this specification is not, and should not be taken as, an acknowledgment or any form of suggestion that this prior art forms part of the common general knowledge in any country.

Over 170 million people are infected with the Hepatitis C virus (HCV) worldwide, resulting in a large disease burden and significant mortality. HCV is rarely cleared in the acute phase of the infection and most patients become chronically infected; a proportion of these patients develop progressive liver disease and fibrosis. The outcome of infection depends on the immune responses of both the innate and cognate immune systems, and these in turn are orchestrated by networks of cytokines and chemokines.

Hepatitis B virus (HBV) also causes debilitating disease conditions and can lead to acute liver failure. HBV is a DNA virus which replicates via an RNA intermediate and utilizes reverse transcription in its replication strategy. The HBV genome is of a complex nature having a partially double-stranded DNA structure with overlapping open reading frames encoding surface, core, polymerase and X genes.

The host virus relationship is a dynamic process in which many viruses such as HCV and HBV attempt to maximize their visibility while the host attempts to prevent and eradicate infection. Inititally, a virus must bind and enter a target cell and migrate to the appropriate cellular compartment in order to replicate and infect other cells. Infected cells may be triggered by the virus to produce cyokines (e.g. TNF-α and IFN-γ) that inhibit one or more stages of the viral replication cycle, thereby limiting the extent of the infection.

Host monocytes and macrophages play a key role in the early response to the virus as they secrete pro-inflammatory cytokines, such as IL-1, TNF-α, IL-6, IL-12 and IL-18 that have indirect and direct effects on the infection. They can recruit further monocytes, natural killer (NK) cells and T-cells to perform functions and they can also help switch the to appropriate Th function to help eradicate the virus.

Innate immunity to microbial pathogens, leading to the production of these pro-inflammatory cytokines, occurs as a result of the activation of Toll Like Receptors (TLRs). The role of TLRs involving bacterial products, e.g. endotoxin and peptidoglycan has recently been clarified (Akashi et al., J Immunol. 164: 3471-3475, 2000; Takeuchi et al., Immunity, 11: 443-451, 1999; Tapping et al., J Immunol. 165: 5780-5787, 2000). More than 10 TLRs have been identified and they play an important role in activation by a number of different bacteria. Recently, this has been extended to viruses with the demonstration that respiratory syncytial virus (RSV) stimulates TLR-4 in a murine model (Kurt-Jones et al., Nat Immunol, 1: 398-401, 2000; Haeberle et al., J Infect Dis. 186: 1199-1206, 2002). In addition, Measles Virus (MV) has been shown to activate TLR-2 dependent signals (Bieback et al., J Virol, 76: 8729-8736, 2002) and double-stranded DNA (the core of many viruses) has been shown to directly mediate responses to through TLR-3 (Matsumoto et al., Biochem Biophys Res Commun. 293: 1364-1369, 2002).

Circulating levels of pro-inflammatory cytokines such as TNF-α are significantly increased in patients with cirrhosis and on-going liver injury (Khoruts et al., Hepatology 13: 267-276, 1991; Tilg et al., Gastroenterology 103: 264-274, 1992; Lee et al., Scand J Gastroenterol 31: 500-505, 1996; Genesca et al., Am J Gastroenterol. 94: 169-177, 1999; von Baehr et al., Gut 47: 281-287, 2000; Andus et al., Hepatology 13: 364-375, 1991; Enomoto et al., J Gastroenterol Hepatol. 15(Suppl): D20-D25, 2000; Neuman et al., J Gastroenterol Hepatol. 17: 196-202, 2002). TNF-α has been shown to be a critical factor in the development of alcohol-induced acute liver injury in animal models (Iimuro et al., Hepatology 26: 1530-1537, 1997; Yin et al., Gastroenterology 117: 942-952, 1999).

Activation of macrophages by endotoxin, a component of the cell walls of Gram-negative bacteria, plays a key role in the pathogenesis of TNF-α over-production and liver injury in such models (Enomoto et al., J. Biomed Sci. 8: 20-27, 2001). Several factors promote endotoxaemia in this setting, including increased translocation of endotoxin from the gut lumen and a reduction in hepatic clearance capacity (Nanji et al., Am J Pathol. 142: 367-373, 1993; Rivera et al., Am J Physiol. 275: G1252-1258, 1998). In the clinical setting, several studies have shown significant, though relatively modest, increases in circulating endotoxin levels in patients with cirrhosis (Khoruts et al., 1991, supra; von Baehr et al., 2000, supra; Fukui et al., J Hepatol. 12: 162-169, 1991; Lin et al., J Hepatol. 22: 165-172, 1995; Chan et al., Scand J Gastroenterol. 32: 942-946, 1997; Hanck et al., Gut 49: 106-111, 2001) and endotoxaemia has been assumed to be responsible for the increased circulating TNF-α levels in this group (Tilg et al., 1992, supra; von Baehr et al., 2000, supra; Schafer et al., Z Gastroenterol. 33: 503-508, 1995; Deviere et al., Gastroenterology 103: 1296-1301, 1992). Endotoxaemia has also been assumed to be responsible for the elevated peripheral blood levels of anti-inflammatory mediators such as soluble TNF receptors (sTNFRs) found in cirrhosis (von Baehr et al., 2000, supra; Tilg et al., Hepatology, 18: 1132-1138, 1993). Nonetheless, a significant correlation between circulating endotoxin and cytokine levels has generally not been demonstrated (Khoruts et al., 1991, supra; Tilg et al., 1993, supra; von Baehr et al., 2000, supra; Chan et al., 1997, supra) raising the possibility that, unlike in animal models, stimuli other than endotoxin may be important.

It has recently been demonstrated that TNF-α production by macrophages in response to microbial stimuli is critically dependent upon activation of TLRs (Medzhitov et al., N Engl J Med. 343: 338-344, 2000; Yoshimura et al., J Immunol. 163: 1-5, 1999; Akira et al., Nature Immunolog, 2: 675-680, 2001). As indicated above, TLRs play a critical role in the induction of innate immunity to microbial pathogens via recognition of conserved molecular patterns. In particular, it is known that TLR-4, in association with CD14, is responsible for signal transduction leading to TNF-α production in response to endotoxin. In contrast, TLR-2 is required for signaling in response to a number of Gram-positive microbial stimuli, including whole bacteria and cell wall components such as peptidoglycan and lipoteichoic acid (LTA) (Medzhitov et al., 2000, supra; Yoshimura et al., 1999; supra; Akira et al., 2001, supra).

There is a need to investigate the role of TLRs in infection with pathogenic entities such as microorganisms or viruses or other diseased states.

### SUMMARY OF THE INVENTION

Throughout this specification, unless the context requires otherwise, the word “comprise”, or variations such as “comprises” or “comprising”, will be understood to imply the inclusion of a stated element or integer or group of elements or integers but not the exclusion of any other element or integer or group of elements or integers.

The present invention identifies cell surface markers which are differentially affected in response to infection by a pathogenic organism or virus or in response to other disease conditions. The cell surface markers are, therefore, useful therapeutic and/or diagnostic targets. In particular, the present invention identifies Toll-like receptors (TLRs) as useful therapeutic and diagnostic markers for infection by pathogenic agents such as microorganisms and viruses or development of other disease conditions. In addition, the TLRs are useful indicators as to the potential responsiveness of a subject to therapeutic intervention including enabling a prediction as to the likelihood or otherwise of a subject responding favourably to therapeutic intervention. Such a prediction is a form of risk assessment. TLRs contain ectodomains with leucine-rich repeats and comprise intracellular motifs which are highly homologous to intracellular signaling domains of interleukin-1 receptor type I (IL-1RI) and IL-1RI accessory protein. Eleven TLRs have so far been identified designated TLR-1 through TLR-11.

In accordance with the present invention, it is identified that TLRs and in particular TLR-2 and TLR-4 are differentially affected on peripheral blood mononuclear cells (PBMCs) and in particular CD14+ PMBC (i.e. monocytes) and liver cells following infection by a pathogenic agent such as a microorganism or virus. TLRs and, in particular, TLR-2 and TLR-4 are, therefore, useful targets for therapeutic or prophylactic agents to treat or help prevent infection by a pathogenic agent. TLRs are also differentially affected in response to other disease conditions such as but not limited to cirrhosis and hepatocellular carcinoma (HCC). They are also useful diagnostic targets to determine whether a subject is or has been infected by a pathogenic entity or whether the subject is predisposed to or has a persistent infection or has another disease condition and can be used as a clinical or epidemiological management tool.

The present invention provides, therefore, therapeutic and/or prophylactic agents capable of modulating levels of TLR, such as TLR-2 and TLR-4. For example, during infection with Hepatitis C virus (HCV), TLR-2 and TLR-4 levels are elevated in PBMCs and in particular monocytes and liver cells. Consequently, therapeutic and prophylactic agents are designed or selected which down-regulate these TLRs. Conversely, infection with Hepatitis B virus (HBV) down-regulates TLR-2. Consequently, therapeutic agents are designed or selected which up-regulate TLR-2 levels. In addition, TLR-2 levels are elevated in liver conditions such as cirrhosis or HCC.

The present invention further provides methods for diagnosis or assessment of infection by a pathogenic agent such as a microorganism or virus by determining the levels of TLRs such as TLR-2 and/or TLR-4 on PBMCs and/or liver cells. In one example, the failure for TLR-2 and/or TLR-4 levels to alter (eg. increase or decrease) during early phase treatment provides an indication that the treatment protocol has some probability of not working.

The present invention contemplates, therefore, therapeutic and diagnostic agents and compositions comprising same useful in the treatment, prophylaxis and/or diagnosis of infection by a pathogenic agent or a predisposition to or persistence of infection in a mammal or avian species. This aspect of the present invention extends to the treatment and diagnosis of other disease conditions such as but not limited to cirrhosis and HCC.

The present invention further provides a method for monitoring a response to therapy as well as determining the efficacy of a therapeutic regimen.

Preferred mammals are humans. Preferred avian species are poultry birds.

### BRIEF DESCRIPTION OF THE FIGURES

FIGS. 1A and 1B are graphical representations showing TLR profiles in control subjects and patients with CHC. Whole blood was stained with directly conjugated antibodies to CD14 and either TLR-2 or TLR-4. Diagrams represent 10,000 CD14-gated cells. The solid line represents expression of the isotype control, the dotted line the control subject and the dashed line the CHC patient.

FIGS. 2A and 2B are graphical representations showing TLR-4 and TLR-2 expression on CD14^{+ve} peripheral blood monocytes in control subjects and patients with HCV infection. TLR-2 and TLR-4 expression was significantly increased in patients with HCV infection.

FIG. 3 is a graphical representation showing plasma endotoxin levels in control subjects and patients with cirrhosis. Box and whisker plots depict the total range, inter-quartile range and median value for each group.

FIG. 4 is a graphical representation showing serum tumor necrosis factor (TNF)-α levels in control subjects and patients with cirrhosis, demonstrating significantly increased values in cirrhotic patients irrespective of aetiology of cirrhosis and in each of the Child-Pugh classes.

FIG. 5 is a graphical representation showing serum soluble TNF-α-receptor I levels in control subjects and patients with cirrhosis, demonstrating significantly increased values in cirrhotic patients irrespective of aetiology of cirrhosis and in each of the Child-Pugh classes.

FIG. 6 is a graphical representation showing serum soluble TNF-α-receptor II levels in control subjects and patients with cirrhosis, demonstrating significantly increased values in cirrhotic patients irrespective of aetiology of cirrhosis and in each of the Child-Pugh classes.

FIG. 7 is a graphical representation showing typical Toll-like receptor (TLR) profiles in control subjects and patients with cirrhosis. Whole blood was stained with directly conjugated antibodies to CD14 and either TLR-2 or TLR-4. Diagrams represent 10,000 CD14-gated cells. The dotted line represents expression of the isotype control, the solid line the control subject and the dashed line the cirrhotic patient.

FIG. 8 is a graphical representation showing toll-like receptor 4 (TLR-4) and TLR-2 expression on CD14^{+ve} peripheral blood monocytes in control subjects and patients with cirrhosis. TLR-4 expression was not significantly different in cirrhotic and control patients (right top panel), irrespective of aetiology of cirrhosis (right middle panel) or Child-Pugh class (right bottom panel). Conversely, TLR-2 expression was significantly increased in patients with cirrhosis (left panel).

FIG. 9 is a graphical representation showing in vitro production of TNF-α PBMCs following stimulation with endotoxin (10 ng/mL) and SEB (10 ng/mL) in control subjects and patients with cirrhosis.

FIG. 10 is a graphical representation showing TLR-2 expression on CD14^{+ve} peripheral blood monocytes in 11 cirrhotic patients before and after oral supplementation for seven days with a Gram-positive gut flora regimen. Supplementation was associated with significantly increased values compared to baseline. Values fell substantially by day 28 post-supplementation in all eight patients in whom such follow-up data could be obtained.

FIG. 11 is a graphical representation of data from 6 patients showing in vitro expression in peripheral blood of TNF-α (FIGS. 11a and b), TLR2 (FIGS. 11c and d) and TLR4 (FIGS. 11e and f) following stimulation with HBV. In which each of b, d and f shows a graphical representation of the average stimulation.

FIG. 12 is a graphical representation showing in vitro expression of TLR2 and TLR 4 in HepG2 cells following transduction with recombinant HBV/baculovirus.

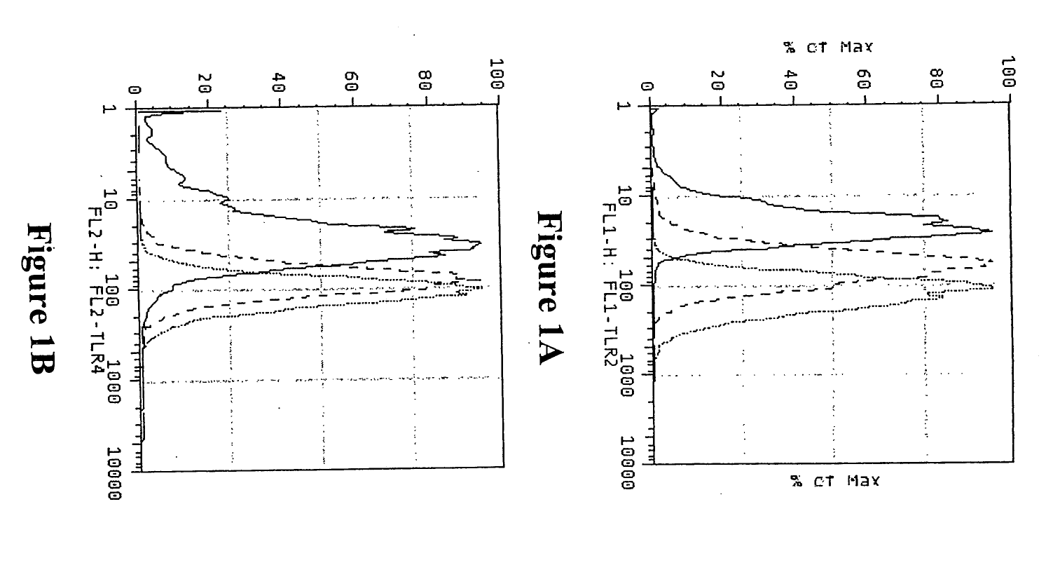

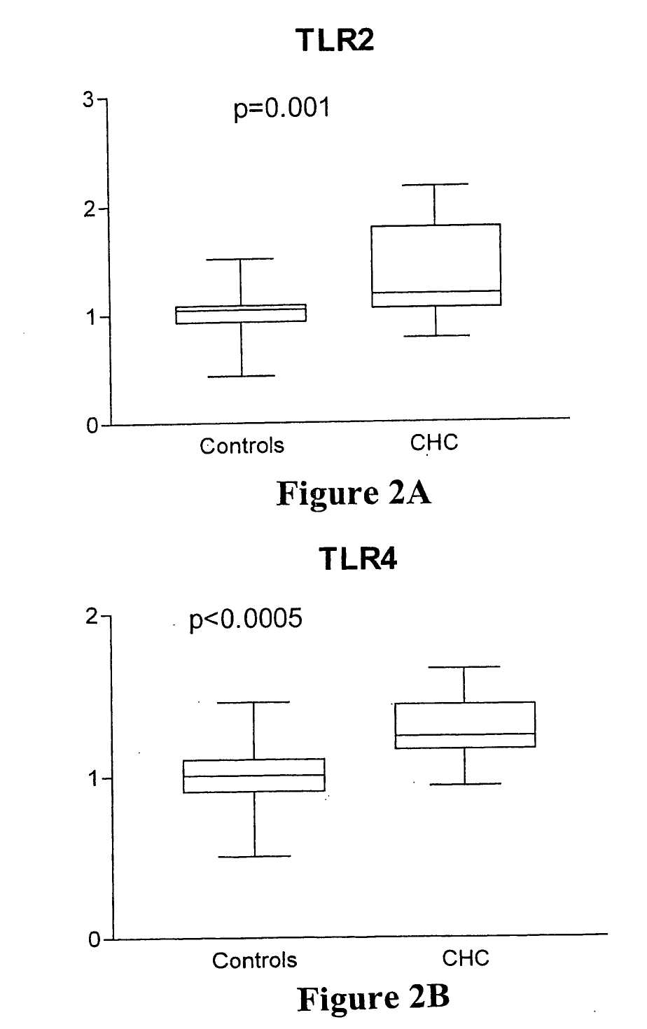

### DETAILED DESCRIPTION OF THE PREFERRED EMBODIMENT

The present invention is predicated in part on the determination that levels of particular cell surface markers correlate with infection by particular pathogenic entities such as microorganisms or viruses or the development of other disease conditions. More particularly, the present invention identifies that TLR genes are differentially expressed during infection by pathogenic entities such as microorganisms or viruses or during the development of certain disease conditions such as cirrhosis and hepatocellular carcinoma (HCC).

In one particular embodiment, infection by HCV results in elevated levels of TLR-2 and TLR-4 in CD14+ PBMCs (i.e. monocytes) and liver cells. The elevated levels of TLR-2 and TLR-4 provide a diagnostic indicator of HCV infection or a predisposition to or persistence of HCV infection. The level of the TLRs are also useful indications of the likelihood that a subject will respond favourably to the therapeutic intervention. Reference to “likelihood” includes making a prediction and making risk assessment of the likelihood or otherwise of success of the treatment protocol. Additionally, TLR-2 and TLR-4 become therapeutic targets for agents which down-regulate TLR-2 and/or TLR-4 levels.

In another embodiment, infection by HBV results in down-regulation of TLR-2 in CD14+ PBMCs and liver cells. Again, this enables the level of TLR-2 to be used as a diagnostic marker for HBV infection and as a target for agents to up-regulate TLR-2 in subjects infected by HBV or who have a predisposition to or persistence of infection with HBV. Normalization of levels of TLR-2 and/or TLR-4 in a prediction through a treatment protocol is working. Comparisons are conveniently made to pretreatment levels or a database of normalized controls.

In yet another embodiment, cirrhosis and HCC induce elevated levels of TLR-2. Consequently, TLR-2 may be used as a diagnostic marker for liver disorders and as a therapeutic target for the treatment of liver disorders.

The present invention provides, therefore, agents which modulate levels of TLRs and in particular TLR-2 and/or TLR-4, diagnostic reagents to determine the levels of TLR-2 and/or TLR-4 and methods for the treatment and/or prophylaxis of infection by a pathogenic organism or virus or development of another disease condition as well as monitoring a therapeutic regimen and determining a subject's predisposition to or persistence of infection by a pathogenic entity or predisposition to another disease condition such as cirrhosis or HCC.

The present invention further contemplates a method for monitoring a response to a therapeutic protocol as well as a means for determining the efficacy of a therapeutic regimen. In particular, the present invention provides a clinical or epidemiological management tool for infection and development of other disease conditions in animals such as mammals and in particular humans.

In a particularly preferred embodiment, the pathogenic entity is a Hepatitis virus such as HBV or HCV. The present invention extends, however, to a range of viruses and microorganisms.

Examples of microorganisms include Salmonella, Escherichia, Klebsiella, Pasteurella, Bacillus (including Bacillus anthracis), Clostridium, Corynebacterium, Mycoplasma, Ureaplasma, Actinomyces, Mycobacterium, Chlamydia, Chlamydophila, Leptospira, Spirochaeta, Borrelia, Treponema, Pseudomonas, Burkholderia, Dichelobacter, Haemophilus, Ralstonia, Xanthomonas, Moraxella, Acinetobacter, Branhamella, Kingella, Erwinia, Enterobacter, Arozona, Citrobacter, Proteus, Providencia, Yersinia, Shigella, Edwardsiella, Vibrio, Rickettsia, Coxiella, Ehrlichia, Arcobacteria, Peptostreptococcus, Candida, Aspergillus, Trichomonas, Bacterioides, Coccidiomyces, Pneumocystis, Cryptosporidium, Porphyromonas, Actinobacillus, Lactococcus, Lactobacillua, Zymononas, Saccharomyces, Propionibacterium, Streptomyces, Penicillum, Neisseria, Staphylococcus, Campylobacter, Streptococcus, Enterococcus or Helicobacter.

Examples of viruses include human immunodeficiency virus (HIV), Varicella-Zoster virus (VZV), herpes simplex virus (HSV), human papillomavirus (HPV), Hepatitis B virus (HBV), Hepatitis A virus (HAV), rhinovirus, echovirus, Coxsackievirus, cytomegalovirus, flavivirus, Ebola virus, paramyxovirus, influenza virus, enterovirus, Epstein-Barr virus, Marburg virus, polio virus, rabies virus, rubella virus, smallpox virus, rubeola virus, vaccina virus, adenovirus or rotavirus.

The preferred liver condition is cirrhosis or HCC.

Accordingly, one aspect of the prevent invention contemplates a method for detecting the presence of infection by a pathogenic agent or a disease condition or a predisposition thereto, said method comprising determining the level of TLR-2 and/or TLR-4 or a homolog thereof wherein an elevated or reduced level of TLR-2 and/or TLR-4 or a homolog thereof is indicative of infection by the pathogenic agent or the presence of the disease condition or predisposition thereto.

Another aspect provides a method for monitoring a response to a therapeutic protocol directed against infection by a pathogenic agent or development of a disease condition said method comprising determining the level of TLR-2 and/or TLR-4 or a homolog thereof wherein the efficacy of the therapeutic response is determined by an elevated or reduced level of TLR-2 and/or TLR-4 or homolog thereof.

Yet another aspect contemplates a method for determining the likelihood that a subject will respond to therapeutic or prophylactic intervention of infection by a pathogenic agent or a disease condition said method comprising determining the level of TLR-2 and/or TLR-4 or a homolog thereof wherein the potential efficacy of the therapeutic intervention is determined by an elevated or reduced level of TLR-2 and/or TLR-4 or a homolog thereof.

Still yet a further aspect provides a method for predicting the outcome of a therapeutic protocol directed against infection by a pathogenic agent or development of a disease condition said method comprising determining the level of TLR-2 and/or TLR-4 or a homolog thereof wherein the efficacy of the therapeutic response is determined by an elevated or reduced level of TLR-2 and/or TLR-4 or homolog thereof.

In accordance with the risk assessment aspects of the present invention, one would expect change in early phase treatment to result in a change or a trend to change levels of TLR-2 and/or TLR-4. When that occurs, the likelihood of successful therapeutic intervention is considered reasonable to high. If there is no trend to alter the TLR levels then this is an indication of a less than successful therapeutic protocol.

It is to be understood that unless otherwise indicated, the subject invention is not limited to specific formulations of components, manufacturing methods, dosage regimens, or the like, as such may vary. It is also to be understood that the terminology used herein is for the purpose of describing particular embodiments only and is not intended to be limiting.

It must be noted that, as used in the subject specification, the singular forms “a,”, “an” and “the” include plural aspects unless the context clearly dictates otherwise. Thus, for example, reference to a “compound” includes a single compound, as well as two or more compounds; reference to “an active agent” includes a single active agent, as well as two or more active agents; and so forth.

In describing and claiming the present invention, the following terminology are used in accordance with the definitions set forth below.

The terms “compound”, “active agent”, “pharmacologically active agent”, “medicament”, “active” and “drug” are used interchangeably herein to refer to a chemical compound that induces a desired pharmacological and/or physiological effect. The terms also encompass pharmaceutically acceptable and pharmacologically active ingredients of those active agents specifically mentioned herein including but not limited to salts, esters, amides, prodrugs, active metabolites, analogs and the like. When the terms “compound”, “active agent”, “pharmacologically active agent”, “medicament”, “active” and “drug” are used, then it is to be understood that this includes the active agent per se as well as pharmaceutically acceptable, pharmacologically active salts, esters, amides, prodrugs, metabolites, analogs, etc. The term “compound” is not to be construed as a chemical compound only but extends to peptides, polypeptides and proteins as well as genetic molecules such as RNA, DNA and chemical analogs thereof. Reference to a “peptide”, “polypeptide” or “protein” includes molecules with a polysaccharide or lipopolysaccharide component. The term “potentiator” is an example of a compound, active agent, pharmacologically active agent, medicament, active and drug which up-regulates the level of a TLR such as TLR-2 or TLR-4. The term “up-regulates” encompasses increasing expression of a TLR gene as well as manipulating a component of the downstream signaling pathway. The term “antagonist” is an example of a compound, active agent, pharmacologically active agent, medicament, active and drug which down-regulates the level of a TLR, such as TLR-2 or TLR-4. Down-regulation may also involve elevating levels of an inhibitor of the TLR signaling pathway.

Accordingly, reference to a TLR also includes reference to the signaling pathway associated with interaction between a ligand and a TLR.

The present invention contemplates, therefore, compounds useful in up-regulating a TLR such as TLR-2 and/or TLR-4 or general or specific TLR signaling. The terms “modulating” or its derivatives, such as “modulate” or “modulation”, are used to describe up- or down-regulation. The compounds have an effect on reducing or preventing or treating infection by a pathogenic organism or virus or treating another disease condition such as cirrhosis or HCC. The preferred cells which carry the TLRs to be modulated include PBMCs and liver cells. A PBMC includes a CD14^{+ve} PMBC and in particular a monocyte and a liver cell includes a hepatocyte. Reference to a “compound”, “active agent”, “pharmacologically active agent”, “medicament”, “active” and “drug” includes combinations of two or more actives such as a potentiator or antagonist of TLR or TLR signaling. A “combination” also includes multi-part such as a two-part pharmaceutical composition where the agents are provided separately and given or dispensed separately or admixed together prior to dispensation.

For example, a multi-part pharmaceutical pack may have a modulator of a TLR and one or more anti-microbial or anti-viral agents.

The terms “effective amount” and “therapeutically effective amount” of an agent as used herein mean a sufficient amount of the agent to provide the desired therapeutic or physiological effect. Furthermore, an “effective TLR-potentiating amount” or an “effective TLR-antagonizing amount” of an agent is a sufficient amount of the agent to directly or indirectly up-regulate or down-regulate the function of a specific TLR such a TLR-2 or TLR-4 or to disrupt or potentiate TLR signaling. This may be accomplished by the agents acting as an agonist or antagonist of the TLR or its signaling components, by the agents which are or mimic components of the TLR signaling pathway, by agents which induce the TLR signaling pathway via other cellular receptors or by the agents antagonizing inhibitors of TLR signaling components. Undesirable effects, e.g. side effects, are sometimes manifested along with the desired therapeutic effect; hence, a practitioner balances the potential benefits against the potential risks in determining what is an appropriate “effective amount”. The exact amount required will vary from subject to subject, depending on the species, age and general condition of the subject, mode of administration and the like. Thus, it may not be possible to specify an exact “effective amount”. However, an appropriate “effective amount” in any individual case may be determined by one of ordinary skill in the art using only routine experimentation.

By “pharmaceutically acceptable” carrier, excipient or diluent is meant a pharmaceutical vehicle comprised of a material that is not biologically or otherwise undesirable, i.e. the material may be administered to a subject along with the selected active agent without causing any or a substantial adverse reaction. Carriers may include excipients and other additives such as diluents, detergents, coloring agents, wetting or emulsifying agents, pH buffering agents, preservatives, and the like. A pharmaceutical composition may also be described depending on the formulation as a vaccine composition.

Similarly, a “pharmacologically acceptable” salt, ester, emide, prodrug or derivative of a compound as provided herein is a salt, ester, amide, prodrug or derivative that this not biologically or otherwise undesirable.

The terms “treating” and “treatment” as used herein refer to reduction in severity and/or frequency of symptoms of infection or disease, elimination of symptoms and/or underlying cause, prevention of the occurrence of symptoms of infection and/or their underlying cause and improvement or remediation of damage. Collateral damage, for example, following viral infection may be liver damage such as cirrhosis or HCC of the liver.

“Treating” a patient may involve prevention of infection or other disease condition or adverse physiological event in a susceptible individual as well as treatment of a clinically symptomatic individual by inhibiting an infection or other disease condition or downstream condition such as liver damage or cancer. Generally, such a condition or disorder is an infection, more particularly, a viral infection and, even more particularly, infection by HBV or HCV. Alternatively, the other disease condition is a liver condition such as cirrhosis or HCC. Thus, for example, the subject method of “treating” a patient with an infection or with a propensity for one to develop encompasses both prevention of the infection or other disease condition as well as treating the infection or other disease condition once established. In any event, the present invention contemplates the treatment or prophylaxis of an infection by a pathogenic organism or virus or the treatment of another disease condition. Pathogenic microorganism may be prokaryotic or eukaryotic organisms or viruses. Examples of prokaryotic organisms include Salmonella, Escherichia, Klebsiella, Pasteurella, Bacillus (including Bacillus anthracis), Clostridium, Corynebacterium, Mycoplasma, Ureaplasma, Actinomyces, Mycobacterium, Chlamydia, Chlamydophila, Leptospira, Spirochaeta, Borrelia, Treponema, Pseudomonas, Burkholderia, Dichelobacter, Haemophilus, Ralstonia, Xanthomonas, Moraxella, Acinetobacter, Branhamella, Kingella, Erwinia, Enterobacter, Arozona, Citrobacter, Proteus, Providencia, Yersinia, Shigella, Edwardsiella, Vibrio, Rickettsia, Coxiella, Ehrlichia, Arcobacteria, Peptostreptococcus, Candida, Aspergillus, Trichomonas, Bacterioides, Coccidiomyces, Pneumocystis, Cryptosporidium, Porphyromonas, Actinobacillus, Lactococcus, Lactobacillua, Zymononas, Saccharomyces, Propionibacterium, Streptomyces, Penicillum, Neisseria, Staphylococcus, Campylobacter, Streptococcus, Enterococcus or Helicobacter. Examples of viruses include human immunodeficiency virus (HIV), Varicella-Zoster virus (VZV), herpes simplex virus (HSV), human papillomavirus (HPV), Hepatitis B virus (HBV), Hepatitis A virus (HAV), rhinovirus, echovirus, Coxsackievirus, cytomegalovirus, flavivirus, Ebola virus, paramyxovirus, influenza virus, enterovirus, Epstein-Barr virus, Marburg virus, polio virus, rabies virus, rubella virus, smallpox virus, rubeola virus, vaccina virus, adenovirus or rotavirus.

Preferably, however, the infection is by HBV or HCV. Reference to “HBV” or “HCV” or their full terms such as “Hepatitis B virus” or “Hepatitis C virus” include all variants including variants resistant to particular therapeutic agents such as nucleoside analogs or immunological agents.

“Patient” as used herein refers to an animal, preferably a mammal and more preferably human who can benefit from the pharmaceutical formulations and methods of the present invention. There is no limitation on the type of animal that could benefit from the presently described pharmaceutical formulations and methods. A patient regardless of whether a human or non-human animal may be referred to as an individual, subject, animal, host or recipient. The compounds and methods of the present invention have applications in human medicine, veterinary medicine as well as in general, domestic or wild animal husbandry. For convenience, an “animal” includes an avian species such as a poultry bird, an aviary bird or game bird. A poultry bird such as a duck is a preferred example of an avian species.

The compounds of the present invention may be large or small molecules, nucleic acid molecules (including antisense or sense molecules), peptides, polypeptides or proteins or hybrid molecules such as RNAi- or siRNA-complexes, ribozymes or DNAzymes. The compounds may need to be modified so as to facilitate entry into a cell. This is not a requirement if the compound interacts with an extracellular receptor. Examples of agents include chemical agents and antibodies which interact with the TLR or genetic molecules which down-regulate or up-regulate expression of a gene encoding a TLR or compound of a TLR signaling pathway.

As indicated above, the preferred animals are humans or other primates, livestock animals, laboratory test animals, companion animals or captive wild animals, as well as avian species.

Examples of laboratory test animals include mice, rats, rabbits, guinea pigs and hamsters. Rabbits and rodent animals, such as rats and mice, provide a convenient test system or animal model. Livestock animals include sheep, cows, pigs, goats, horses and donkeys. Non-mammalian animals such as avian species (such as ducks), zebrafish, amphibians (including cane toads) and Drosophila species such as Drosophila melanogaster are also contemplated.

The present invention provides, therefore, agents which antagonize or agonize (ie. potentiate or activate) TLRs such as TLR-2 and/or TLR-4.

The present invention contemplates methods of screening for such agents comprising, for example, contacting a candidate drug with a TLR such as TLR-2 or TLR-4 or a part thereof. Such a TLR molecule is referred to herein as a “target” or “target molecule”. The screening procedure includes assaying (i) for the presence of a complex between the drug and the target, or (ii) an alteration in the expression levels of nucleic acid molecules encoding the target. One form of assay involves competitive binding assays. In such competitive binding assays, the target is typically labeled. Free target is separated from any putative complex and the amount of free (i.e. uncomplexed) label is a measure of the binding of the agent being tested to target molecule. One may also measure the amount of bound, rather than free, target. It is also possible to label the compound rather than the target and to measure the amount of compound binding to target in the presence and in the absence of the drug being tested. Such compounds may inhibit the target which is useful, for example, in finding inhibitors of TLR-2 and/or TLR-4 required for treating HCV infection or other disease condition or may protect TLR-2 or other components from being inhibited which would be required for treating HBV infection.

Another technique for drug screening provides high throughput screening for compounds having suitable binding affinity to a target and is described in detail in Geysen (International Patent Publication No. WO 84/03564). Briefly stated, large numbers of different small peptide test compounds are synthesized on a solid substrate, such as plastic pins or some other surface. The peptide test compounds are reacted with a target and washed. Bound target molecule is then detected by methods well known in the art. This method may be adapted for screening for non-peptide, chemical entities. This aspect, therefore, extends to combinatorial approaches to screening for target antagonists or agonists of TLRs such as TLR-2 or TLR-4.

Purified target can be coated directly onto plates for use in the aforementioned drug screening techniques. However, non-neutralizing antibodies to the target may also be used to immobilize the target on the solid phase. Antibodies specific for a TLR, such as TLR-2 or TLR-4, may also be useful as inhibitors of these TLRs such as in the treatment of HCV infection or other disease condition.

The present invention also contemplates the use of competitive drug screening assays in which neutralizing antibodies capable of specifically binding the target compete with a test compound for binding to the target or fragments thereof. In this manner, the antibodies can be used to detect the presence of any peptide which shares one or more antigenic determinants of the target.

Antibodies to a TLR may be polyclonal or monoclonal although monoclonal antibodies are preferred. Antibodies may be prepared by any of a number of means. For the detection of a TLR, antibodies are generally but not necessarily derived from non-human animals such as primates, livestock animals (e.g. sheep, cows, pigs, goats, horses), laboratory test animals (e.g. mice, rats, guinea pigs, rabbits) and companion animals (e.g. dogs, cats). Generally, antibody based assays are conducted in vitro on cell or tissue biopsies. However, if an antibody is suitably deimmunized or, in the case of human use, humanized, then the antibody can be labeled with, for example, a nuclear tag, administered to a subject and the site of nuclear label accumulation determined by radiological techniques. The TLR antibody is regarded, therefore, as a pathogenic marker targeting agent. Accordingly, the present invention extends to deimmunized forms of the antibodies for use in pathogenic target imaging in human and non-human subjects. This is described further below.

For the generation of antibodies to TLR, the enzyme is required to be extracted from a biological sample whether this be from animal including human tissue or from cell culture if produced by recombinant means. Generally, monocytes and hepatocytes are a convenient source. The TLR can be separated from the biological sample by any suitable means. For example, the separation may take advantage of any one or more of TLR's surface charge properties, size, density, biological activity and its affinity for another entity (e.g. another protein or chemical compound to which it binds or otherwise associates). Thus, for example, separation of TLR from the biological sample may be achieved by any one or more of ultra-centrifugation, ion-exchange chromatography (e.g. anion exchange chromatography, cation exchange chromatography), electrophoresis (e.g. polyacrylamide gel electrophoresis, isoelectric focussing), size separation (e.g., gel filtration, ultra-filtration) and affinity-mediated separation (e.g. immunoaffrnity separation including, but not limited to, magnetic bead separation such as Dynabead (trademark) separation, immunochromatography, immuno-precipitation). Choice of the separation technique(s) employed may depend on the biological activity or physical properties of the particular TLR sought or from which tissues it is obtained.

Preferably, the separation of TLR from the biological fluid preserves conformational epitopes present on the kinase and, thus, suitably avoids techniques that cause denaturation of the enzyme. Persons of skill in the art will recognize the importance of maintaining or mimicking as close as possible physiological conditions peculiar to the TLR (e.g. the biological sample from which it is obtained) to ensure that the antigenic determinants or active site/s on the TLR, which are exposed to the animal, are structurally identical to that of the native enzyme. This ensures the raising of appropriate antibodies in the immunized animal that would recognize the native enzyme.

Immunization and subsequent production of monoclonal antibodies can be carried out using standard protocols as for example described by Köhler and Milstein (Kohler and Milstein, Nature 256: 495-499, 1975; Kohler and Milstein, Eur. J. Immunol. 6(7): 511-519, 1976), Coligan et al. (“Current Protocols in Immunology, John Wiley & Sons, Inc., 1991-1997) or Toyama et al. (Monoclonal Antibody, Experiment Manual”, published by Kodansha Scientific, 1987). Essentially, an animal is immunized with a TLR or a sample comprising a TLR by standard methods to produce antibody-producing cells, particularly antibody-producing somatic cells (e.g. B lymphocytes). These cells can then be removed from the immunized animal for immortalization.

Where a fragment of TLR is used to generate antibodies, it may need to first be associated with a carrier. By “carrier” is meant any substance of typically high molecular weight to which a non- or poorly immunogenic substance (e.g. a hapten) is naturally or artificially linked to enhance its immunogenicity.

Immortalization of antibody-producing cells may be carried out using methods which are well-known in the art. For example, the immortalization may be achieved by the transformation method using Epstein-Barr virus (EBV) (Kozbor et al., Methods in Enzymology 121: 140, 1986). In a preferred embodiment, antibody-producing cells are immortalized using the cell fusion method (described in Coligan et al., 1991-1997, supra), which is widely employed for the production of monoclonal antibodies. In this method, somatic antibody-producing cells with the potential to produce antibodies, particularly B cells, are fused with a myeloma cell line. These somatic cells may be derived from the lymph nodes, spleens and peripheral blood of primed animals, preferably rodent animals such as mice and rats. Mice spleen cells are particularly useful. It would be possible, however, to use rat, rabbit, sheep or goat cells, or cells from other animal species instead.

Specialized myeloma cell lines have been developed from lymphocytic tumours for use in hybridoma-producing fusion procedures (Kohler and Milstein, 1976, supra; Shulman et al., Nature 276: 269-270, 1978; Volk et al., J. Virol. 42(1): 220-227, 1982). These cell lines have been developed for at least three reasons. The first is to facilitate the selection of fused hybridomas from unfused and similarly indefinitely self-propagating myeloma cells. Usually, this is accomplished by using myelomas with enzyme deficiencies that render them incapable of growing in certain selective media that support the growth of hybridomas. The second reason arises from the inherent ability of lymphocytic tumour cells to produce their own antibodies. To eliminate the production of tumour cell antibodies by the hybridomas, myeloma cell lines incapable of producing endogenous light or heavy immunoglobulin chains are used. A third reason for selection of these cell lines is for their suitability and efficiency for fusion.

Many myeloma cell lines may be used for the production of fused cell hybrids, including, e.g. P3X63-Ag8, P3X63-AG8.653, P3/NS1-Ag4-1 (NS-1), Sp2/0-Ag14 and S194/5.XXO.Bu.1. The P3X63-Ag8 and NS-1 cell lines have been described by Kbhler and Milstein (1976, supra). Shulman et al. (1978, supra) developed the Sp2/0-Ag14 myeloma line. The S194/5.XXO.Bu.1 line was reported by Trowbridge (J. Exp. Med. 148(1): 313-323, 1978).

Methods for generating hybrids of antibody-producing spleen or lymph node cells and myeloma cells usually involve mixing somatic cells with myeloma cells in a 10:1 proportion (although the proportion may vary from about 20:1 to about 1:1), respectively, in the presence of an agent or agents (chemical, viral or electrical) that promotes the fusion of cell membranes. Fusion methods have been described (Kohler and Milstein, 1975, supra; Kohler and Milstein, 1976, supra; Gefter et al., Somatic Cell Genet. 3: 231-236, 1977; Volk et al., 1982, supra). The fusion-promoting agents used by those investigators were Sendai virus and polyethylene glycol (PEG).

Because fusion procedures produce viable hybrids at very low frequency (e.g. when spleens are used as a source of somatic cells, only one hybrid is obtained for roughly every 1×10⁵ spleen cells), it is preferable to have a means of selecting the fused cell hybrids from the remaining unfused cells, particularly the unfused myeloma cells. A means of detecting the desired antibody-producing hybridomas among other resulting fused cell hybrids is also necessary. Generally, the selection of fused cell hybrids is accomplished by culturing the cells in media that support the growth of hybridomas but prevent the growth of the unfused myeloma cells, which normally would go on dividing indefinitely. The somatic cells used in the fusion do not maintain long-term viability in in vitro culture and hence do not pose a problem. In the example of the present invention, myeloma cells lacking hypoxanthine phosphoribosyl transferase (HPRT-negative) were used. Selection against these cells is made in hypoxanthine/aminopterin/thymidine (HAT) medium, a medium in which the fused cell hybrids survive due to the HPRT-positive genotype of the spleen cells. The use of myeloma cells with different genetic deficiencies (drug sensitivities, etc.) that can be selected against in media supporting the growth of genotypically competent hybrids is also possible.

Several weeks are required to selectively culture the fused cell hybrids. Early in this time period, it is necessary to identify those hybrids which produce the desired antibody, so that they may subsequently be cloned and propagated. Generally, around 10% of the hybrids obtained produce the desired antibody, although a range of from about 1 to about 30% is not uncommon. The detection of antibody-producing hybrids can be achieved by any one of several standard assay methods, including enzyme-linked immunoassay and radioimmunoassay techniques as, for example, described in Kennet et al. (Monoclonal Antibodies and Hybridomas: A New Dimension in Biological Analyses, pp 376-384, Plenum Press, New York, 1980) and by FACS analysis (O'Reilly et al., Biotechniques 25: 824-830, 1998).

Once the desired fused cell hybrids have been selected and cloned into individual antibody-producing cell lines, each cell line may be propagated in either of two standard ways. A suspension of the hybridoma cells can be injected into a histocompatible animal. The injected animal will then develop tumours that secrete the specific monoclonal antibody produced by the fused cell hybrid. The body fluids of the animal, such as serum or ascites fluid, can be tapped to provide monoclonal antibodies in high concentration. Alternatively, the individual cell lines may be propagated in vitro in laboratory culture vessels. The culture medium containing high concentrations of a single specific monoclonal antibody can be harvested by decantation, filtration or centrifugation, and subsequently purified.

The cell lines are tested for their specificity to detect the TLR of interest by any suitable immunodetection means. For example, cell lines can be aliquoted into a number of wells and incubated and the supernatant from each well is analyzed by enzyme-linked immunosorbent assay (ELISA), indirect fluorescent antibody technique, or the like. The cell line(s) producing a monoclonal antibody capable of recognizing the target TLR but which does not recognize non-target epitopes are identified and then directly cultured in vitro or injected into a histocompatible animal to form tumours and to produce, collect and purify the required antibodies.

These antibodies are TLR-specific. This means that the antibodies are capable of distinguishing a particular TLR from other molecules. More broad spectrum antibodies may be used provided that they do not cross-react with molecules in a normal cell.

Where the monoclonal antibody is destined for use as a therapeutic agent such as to inhibit TLR-2 or TLR-4, then, it will need to be deimmunized with respect to the host into which it will be introduced (e.g. a human). The deimmunization process may take any of a number of forms including the preparation of chimeric antibodies which have the same or similar specificity as the monoclonal antibodies prepared according to the present invention. Chimeric antibodies are antibodies whose light and heavy chain genes have been constructed, typically by genetic engineering, from immunoglobulin variable and constant region genes belonging to different species. Thus, in accordance with the present invention, once a hybridoma producing the desired monoclonal antibody is obtained, techniques are used to produce interspecific monoclonal antibodies wherein the binding region of one species is combined with a non-binding region of the antibody of another species (Liu et al., Proc. Natl. Acad. Sci. USA 84: 3439-3443, 1987). For example, complementary determining regions (CDRs) from a non-human (e.g. murine) monoclonal antibody can be grafted onto a human antibody, thereby “humanizing” the murine antibody (European Patent No. 0 239 400; Jones et al., Nature 321: 522-525, 1986; Verhoeyen et al., Science 239: 1534-1536, 1988; Richmann et al., Nature 332: 323-327, 1988). In this case, the deimmunizing process is specific for humans. More particularly, the CDRs can be grafted onto a human antibody variable region with or without human constant regions. The non-human antibody providing the CDRs is typically referred to as the “donor” and the human antibody providing the framework is typically referred to as the “acceptor”. Constant regions need not be present, but if they are, they must be substantially identical to human immunoglobulin constant regions, i.e. at least about 85-90%, preferably about 95% or more identical. Hence, all parts of a humanized antibody, except possibly the CDRs, are substantially identical to corresponding parts of natural human immunoglobulin sequences. Thus, a “humanized antibody” is an antibody comprising a humanized light chain and a humanized heavy chain immunoglobulin. A donor antibody is said to be “humanized”, by the process of “humanization”, because the resultant humanized antibody is expected to bind to the same antigen as the donor antibody that provides the CDRs. Reference herein to “humanized” includes reference to an antibody deimmunized to a particular host, in this case, a human host.

It will be understood that the deimmunized antibodies may have additional conservative amino acid substitutions which have substantially no effect on antigen binding or other immunoglobulin functions. Exemplary conservative substitutions may be made according to Table 1.

TABLE 1

| ORIGINAL RESIDUE | EXEMPLARY SUBSTITUTIONS |
|---|---|
| Ala | Ser |
| Arg | Lys |
| Asn | Gln, His |
| Asp | Glu |
| Cys | Ser |
| Gln | Asn |
| Glu | Asp |
| Gly | Pro |
| His | Asn, Gln |
| Ile | Leu, Val |
| Leu | Ile, Val |
| Lys | Arg, Gln, Glu |
| Met | Leu, Ile |
| Phe | Met, Leu, Tyr |
| Ser | Thr |
| Thr | Ser |
| Tip | Tyr |
| Tyr | Trp, Phe |
| Val | Ile, Leu |

Exemplary methods which may be employed to produce deimmunized antibodies according to the present invention are described, for example, in Richmann et al., 1988, supra; European Patent No. 0 239 400; U.S. Pat. No. 6,056,957, U.S. Pat. No. 6,180,370, U.S. Pat. No. 6,180,377.

Thus, in one embodiment, the present invention contemplates a deimmunized antibody molecule having specificity for an epitope recognized by a monoclonal antibody to a TLR such as TLR-2 or TLR-4 wherein at least one of the CDRs of the variable domain of said deimmunized antibody is derived from the said monoclonal antibody to said TLR and the remaining immunoglobulin-derived parts of the deimmunized antibody molecule are derived from an immunoglobulin or an analog thereof from the host for which the antibody is to be deimmunized.

This aspect of the present invention involves manipulation of the framework region of a non-human antibody.

The present invention extends to mutants and derivatives of the subject antibodies but which still retain specificity for TLR.

The terms “mutant” or “derivatives” includes one or more amino acid substitutions, additions and/or deletions.

As used herein, the term “CDR” includes CDR structural loops which covers to the three light chain and the three heavy chain regions in the variable portion of an antibody framework region which bridge P strands on the binding portion of the molecule. These loops have characteristic canonical structures (Chothia et al., J. Mol. Biol. 196: 901, 1987; Chothia et al., J. Mol. Biol. 227: 799, 1992).

By “framework region” is meant region of an immunoglobulin light or heavy chain variable region, which is interrupted by three hypervariable regions, also called CDRs. The extent of the framework region and CDRs have been precisely defined (see, for example, Kabat et al., “Sequences of Proteins of Immunological Interest”, U.S. Department of Health and Human Sciences, 1983). The sequences of the framework regions of different light or heavy chains are relatively conserved within a species. As used herein, a “human framework region” is a framework region that is substantially identical (about 85% or more, usually 90-95% or more) to the framework region of a naturally occurring human immunoglobulin. The framework region of an antibody, that is the combined framework regions of the constituent light and heavy chains, serves to position and align the CDRs. The CDRs are primarily responsible for binding to an epitope of the TLR.

As used herein, the term “heavy chain variable region” means a polypeptide which is from about 110 to 125 amino acid residues in length, the amino acid sequence of which corresponds to that of a heavy chain of a monoclonal antibody of the invention, starting from the amino-terminal (N-terminal) amino acid residue of the heavy chain. Likewise, the term “light chain variable region” means a polypeptide which is from about 95 to 130 amino acid residues in length, the amino acid sequence of which corresponds to that of a light chain of a monoclonal antibody of the invention, starting from the N-terminal amino acid residue of the light chain. Full-length immunoglobulin “light chains” (about 25 Kd or 214 amino acids) are encoded by a variable region gene at the NH₂-terminus (about 110 amino acids) and a κ or λ constant region gene at the COOH-terminus. Full-length immunoglobulin “heavy chains” (about 50 Kd or 446 amino acids), are similarly encoded by a variable region gene (about 116 amino acids) and one of the other aforementioned constant region genes, e.g. γ (encoding about 330 amino acids).

The term “immunoglobulin” or “antibody” is used herein to refer to a protein consisting of one or more polypeptides substantially encoded by immunoglobulin genes. The recognized immunoglobulin genes include the κ, λ, α, γ (IgG₁, IgG₂, IgG₃, IgG₄), δ, ε and μ constant region genes, as well as the myriad immunoglobulin variable region genes. One form of immunoglobulin constitutes the basic structural unit of an antibody. This form is a tetramer and consists of two identical pairs of immunoglobulin chains, each pair having one light and one heavy chain. In each pair, the light and heavy chain variable regions are together responsible for binding to an antigen, and the constant regions are responsible for the antibody effector functions. In addition to antibodies, immunoglobulins may exist in a variety of other forms including, for example, Fv, Fab, Fab′ and (Fab′)₂.

The present invention also contemplates the use and generation of fragments of monoclonal antibodies produced by the method of the present invention including, for example, Fv, Fab, Fab′ and F(ab′)₂ fragments. Such fragments may be prepared by standard methods as for example described by Coligan et al. (1991-1997, supra).

The present invention also contemplates synthetic or recombinant antigen-binding molecules with the same or similar specificity as the monoclonal antibodies of the invention. Antigen-binding molecules of this type may comprise a synthetic stabilized Fv fragment. Exemplary fragments of this type include single chain Fv fragments (sFv, frequently termed scFv) in which a peptide linker is used to bridge the N terminus or C terminus of a V_{H} domain with the C terminus or N-terminus, respectively, of a V_{L} domain. ScFv lack all constant parts of whole antibodies and are not able to activate complement. Suitable peptide linkers for joining the V_{H} and V_{L} domains are those which allow the V_{H} and V_{L} domains to fold into a single polypeptide chain having an antigen binding site with a three dimensional structure similar to that of the antigen binding site of a whole antibody from which the Fv fragment is derived. Linkers having the desired properties may be obtained by the method disclosed in U.S. Pat. No. 4,946,778. However, in some cases a linker is absent. ScFvs may be prepared, for example, in accordance with methods outlined in Krebber et al. (J. Immunol. Methods 201(1): 35-55, 1997). Alternatively, they may be prepared by methods described in U.S. Pat. No 5,091,513, European Patent No. 239,400 or the articles by Winter and Milstein (Nature 349: 293, 1991) and Plückthun et al. (In Antibody engineering: A practical approach, 203-252, 1996).

Alternatively, the synthetic stabilized Fv fragment comprises a disulphide stabilized Fv (dsFv) in which cysteine residues are introduced into the V_{H} and V_{L} domains such that in the fully folded Fv molecule the two residues will form a disulphide bond therebetween. Suitable methods of producing dsFv are described, for example, in (Glockshuber et al., Biochem. 29: 1363-1367, 1990; Reiter et al., J. Biol. Chem. 269: 18327-18331, 1994; Reiter et al., Biochem. 33: 5451-5459, 1994; Reiter et al., Cancer Res. 54: 2714-2718, 1994; Webber et al., Mol. Immunol. 32: 249-258, 1995).

Also contemplated as synthetic or recombinant antigen-binding molecules are single variable region domains (termed Dabs) as, for example, disclosed in (Ward et al., Nature 341: 544-546, 1989; Hamers-Casterman et al., Nature 363: 446-448, 1993; Davies & Riechmann, FEBS Lett. 339: 285-290, 1994).

Alternatively, the synthetic or recombinant antigen-binding molecule may comprise a “minibody”. In this regard, minibodies are small versions of whole antibodies, which encode in a single chain the essential elements of a whole antibody. Suitably, the minibody is comprised of the V_{H} and V_{L} domains of a native antibody fused to the hinge region and CH3 domain of the immunoglobulin molecule as, for example, disclosed in U.S. Pat. No. 5,837,821.

In an alternate embodiment, the synthetic or recombinant antigen binding molecule may comprise non-immunoglobulin derived, protein frameworks. For example, reference may be made to (Ku & Schutz, Proc. Natl. Acad. Sci. USA 92: 6552-6556, 1995) which discloses a four-helix bundle protein cytochrome b562 having two loops randomized to create CDRs, which have been selected for antigen binding.

The synthetic or recombinant antigen-binding molecule may be multivalent (i.e. having more than one antigen binding site). Such multivalent molecules may be specific for one or more antigens. Multivalent molecules of this type may be prepared by dimerization of two antibody fragments through a cysteinyl-containing peptide as, for example disclosed by (Adams et al., Cancer Res. 53: 4026-4034, 1993; Cumber et al., J. Immunol. 149: 120-126, 1992). Alternatively, dimerization may be facilitated by fusion of the antibody fragments to amphiphilic helices that naturally dimerize (Plünckthun, Biochem 31: 1579-1584, 1992) or by use of domains (such as leucine zippers jun and fos) that preferentially heterodimerize (Kostelny et al., J. Immunol. 148: 1547-1553, 1992). Multivalent antibodies are useful, for example, in detecting different forms of TLRs such as TLR-2 and TLR-4.

Yet another useful source of compounds useful in modulating TLR activity is a chemically modified ligand such as a cytokine or other activator of TLR which may then in turn activate or inhibit a TLR pathway.

In addition, compounds can be selected which interrupt or antagonize or agonize the interaction between a TLR and its ligand.

Analogs of proteinaceous molecules (e.g. ligands of a TLR) contemplated herein include but are not limited to modification to side chains, incorporating of unnatural amino acids and/or their derivatives during peptide, polypeptide or protein synthesis and the use of crosslinkers and other methods which impose conformational constraints on the proteinaceous molecule or their analogs.

Examples of side chain modifications contemplated by the present invention include modifications of amino groups such as by reductive alkylation by reaction with an aldehyde followed by reduction with NaBH₄; amidination with methylacetimidate; acylation with acetic anhydride; carbamoylation of amino groups with cyanate; trinitrobenzylation of amino groups with 2,4,6-trinitrobenzene sulphonic acid (TNBS); acylation of amino groups with succinic anhydride and tetrahydrophthalic anhydride; and pyridoxylation of lysine with pyridoxal-5-phosphate followed by reduction with NaBH₄.

The guanidine group of arginine residues may be modified by the formation of heterocyclic condensation products with reagents such as 2,3-butanedione, phenylglyoxal and glyoxal.

The carboxyl group may be modified by carbodiimide activation via O-acylisourea formation followed by subsequent derivitization, for example, to a corresponding amide.

Sulphydryl groups may be modified by methods such as carboxymethylation with iodoacetic acid or iodoacetamide; performic acid oxidation to cysteic acid; formation of a mixed disulphides with other thiol compounds; reaction with maleimide, maleic anhydride or other substituted maleimide; formation of mercurial derivatives using 4-chloromercuribenzoate, 4-chloromercuriphenylsulphonic acid, phenylmercury chloride, 2-chloromercuri-4-nitrophenol and other mercurials; carbamoylation with cyanate at alkaline pH.

Tryptophan residues may be modified by, for example, oxidation with N-bromosuccinimide or alkylation of the indole ring with 2-hydroxy-5-nitrobenzyl bromide or sulphenyl halides. Tyrosine residues on the other hand, may be altered by nitration with tetranitromethane to form a 3-nitrotyrosine derivative.

Modification of the imidazole ring of a histidine residue may be accomplished by alkylation with iodoacetic acid derivatives or N-carbethoxylation with diethylpyrocarbonate.

Examples of incorporating unnatural amino acids and derivatives during peptide synthesis include, but are not limited to, use of norleucine, 4-amino butyric acid, 4-amino-3-hydroxy-5-phenylpentanoic acid, 6-aminohexanoic acid, t-butylglycine, norvaline, phenylglycine, ornithine, sarcosine, 4-amino-3-hydroxy-6-methylheptanoic acid, 2-thienyl alanine and/or D-isomers of amino acids. A list of unnatural amino acid, contemplated herein is shown in Table 2.

TABLE 2
Codes for non-conventional amino acids

| Non-conventional amino acid | Code |
|---|---|
| α-aminobutyric acid | Abu |
| α-amino-α-methylbutyrate | Mgabu |
| aminocyclopropane-carboxylate | Cpro |
| aminoisobutyric acid | Aib |
| aminonorbornyl-carboxylate | Norb |
| cyclohexylalanine | Chexa |
| cyclopentylalanine | Cpen |
| D-alanine | Dal |
| D-arginine | Darg |
| D-aspartic acid | Dasp |
| D-cysteine | Dcys |
| D-glutamine | Dgln |
| D-glutamic acid | Dglu |
| D-histidine | Dhis |
| D-isoleucine | Dile |
| D-leucine | Dleu |
| D-lysine | Dlys |
| D-methionine | Dmet |
| D-ornithine | Dorn |
| D-phenylalanine | Dphe |
| D-proline | Dpro |
| D-serine | Dser |
| D-threonine | Dthr |
| D-tryptophan | Dtrp |
| D-tyrosine | Dtyr |
| D-valine | Dval |
| D-α-methylalanine | Dmala |
| D-α-methylarginine | Dmarg |
| D-α-methylasparagine | Dmasn |
| D-α-methylaspartate | Dmasp |
| D-α-methylcysteine | Dmcys |
| D-α-methylglutamine | Dmgln |
| D-α-methylhistidine | Dmhis |
| D-α-methylisoleucine | Dmile |
| D-α-methylleucine | Dmleu |
| D-α-methyllysine | Dmlys |
| D-α-methylmethionine | Dmmet |
| D-α-methylornithine | Dmorn |
| D-α-methylphenylalanine | Dmphe |
| D-α-methylproline | Dmpro |
| D-α-methylserine | Dmser |
| D-α-methylthreonine | Dmthr |
| D-α-methyltryptophan | Dmtrp |
| D-α-methyltyrosine | Dmty |
| D-α-methylvaline | Dmval |
| D-N-methylalanine | Dnmala |
| D-N-methylarginine | Dnmarg |
| D-N-methylasparagine | Dnmasn |
| D-N-methylaspartate | Dnmasp |
| D-N-methylcysteine | Dnmcys |
| D-N-methylglutamine | Dnmgln |
| D-N-methylglutamate | Dnmglu |
| D-N-methylhistidine | Dnmhis |
| D-N-methylisoleucine | Dnmile |
| D-N-methylleucine | Dnmleu |
| D-N-methyllysine | Dmnlys |
| N-methylcyclohexylalanine | Nmchexa |
| D-N-methylornithine | Dnmorn |
| N-methylglycine | Nala |
| N-methylaminoisobutyrate | Nmaib |
| N-(1-methylpropyl)glycine | Nile |
| N-(2-methylpropyl)glycine | Nleu |
| D-N-methyltryptophan | Dnmtrp |
| D-N-methyltyrosine | Dnmtyr |
| D-N-methylvaline | Dnmval |
| γ-aminobutyric acid | Gabu |
| L-t-butylglycine | Tbug |
| L-ethylglycine | Etg |
| L-homophenylalanine | Hphe |
| L-α-methylarginine | Marg |
| L-α-methylaspartate | Masp |
| L-α-methylcysteine | Mcys |
| L-α-methylglutamine | Mgln |
| L-α-methylhistidine | Mhis |
| L-α-methylisoleucine | Mile |
| L-α-methylleucine | Mleu |
| L-α-methylmethionine | Mmet |
| L-α-methylnorvaline | Mnva |
| L-α-methylphenylalanine | Mphe |
| L-α-methylserine | Mser |
| L-α-methyltryptophan | Mtrp |
| L-α-methylvaline | Mval |
| N-(N-(2,2-diphenylethyl)carbamylmethyl)glycine | Nnbhm |
| 1-carboxy-1-(2,2-diphenylethylamino)cyclopropane | Nmbc |
| L-N-methylalanine | Nmala |
| L-N-methylarginine | Nmarg |
| L-N-methylasparagine | Nmasn |
| L-N-methylaspartic acid | Nmasp |
| L-N-methylcysteine | Nmcys |
| L-N-methylglutamine | Nmgln |
| L-N-methylglutamic acid | Nmglu |
| L-Nmethylhistidine | Nmhis |
| L-N-methylisolleucine | Nmile |
| L-N-methylleucine | Nmleu |
| L-N-methyllysine | Nmlys |
| L-N-methylmethionine | Nmmet |
| L-N-methylnorleucine | Nmnle |
| L-N-methylnorvaline | Nmnva |
| L-N-methylornithine | Nmorn |
| L-N-methylphenylalanine | Nmphe |
| L-N-methylproline | Nmpro |
| L-N-methylserine | Nmser |
| L-N-methylthreonine | Nmthr |
| L-N-methyltryptophan | Nmtrp |
| L-N-methyltyrosine | Nmtyr |
| L-N-methylvaline | Nmval |
| L-N-methylethylglycine | Nmetg |
| L-N-methyl-t-butylglycine | Nmtbug |
| L-norleucine | Nle |
| L-norvaline | Nva |
| α-methyl-aminoisobutyrate | Maib |
| α-methyl-γ-aminobutyrate | Mgabu |
| α-methylcyclohexylalanine | Mchexa |
| α-methylcylcopentylalanine | Mcpen |
| α-methyl-α-napthylalanine | Manap |
| α-methylpenicillamine | Mpen |
| N-(4-aminobutyl)glycine | Nglu |
| N-(2-aminoethyl)glycine | Naeg |
| N-(3-aminopropyl)glycine | Norn |
| N-amino-α-methylbutyrate | Nmaabu |
| α-napthylalanine | Anap |
| N-benzylglycine | Nphe |
| N-(2-carbamylethyl)glycine | Ngln |
| N-(carbamylmethyl)glycine | Nasn |
| N-(2-carboxyethyl)glycine | Nglu |
| N-(carboxymethyl)glycine | Nasp |
| N-cyclobutylglycine | Ncbut |
| N-cycloheptylglycine | Nchep |
| N-cyclohexylglycine | Nchex |
| N-cyclodecylglycine | Ncdec |
| N-cylcododecylglycine | Ncdod |
| N-cyclooctylglycine | Ncoct |
| N-cyclopropylglycine | Ncpro |
| N-cycloundecylglycine | Ncund |
| N-(2,2-diphenylethyl)glycine | Nbhm |
| N-(3,3-diphenylpropyl)glycine | Nbhe |
| N-(3-guanidinopropyl)glycine | Narg |
| N-(1-hydroxyethyl)glycine | Nthr |
| N-(hydroxyethyl))glycine | Nser |
| N-(imidazolylethyl))glycine | Nhis |
| N-(3-indolylyethyl)glycine | Nhtrp |
| N-methyl-γ-aminobutyrate | Nmgabu |
| D-N-methylmethionine | Dnmmet |
| N-methylcyclopentylalanine | Nmcpen |
| D-N-methylphenylalanine | Dnmphe |
| D-N-methylproline | Dnmpro |
| D-N-methylserine | Dnmser |
| D-N-methylthreonine | Dnmthr |
| N-(1-methylethyl)glycine | Nval |
| N-methyla-napthylalanine | Nmanap |
| N-methylpenicillamine | Nmpen |
| N-(p-hydroxyphenyl)glycine | Nhtyr |
| N-(thiomethyl)glycine | Ncys |
| penicillamine | Pen |
| L-α-methylalanine | Mala |
| L-α-methylasparagine | Masn |
| L-α-methyl-t-butylglycine | Mtbug |
| L-methylethylglycine | Metg |
| L-α-methylglutamate | Mglu |
| L-α-methylhomophenylalanine | Mhphe |
| N-(2-methylthioethyl)glycine | Nmet |
| L-α-methyllysine | Mlys |
| L-α-methylnorleucine | Mnle |
| L-α-methylornithine | Morn |
| L-α-methylproline | Mpro |
| L-α-methylthreonine | Mthr |
| L-α-methyltyrosine | Mtyr |
| L-N-methylhomophenylalanine | Nmhphe |
| N-(N-(3,3-diphenylpropyl)carbamylmethyl)glycine | Nnbhe |

Crosslinkers can be used, for example, to stabilize 3D conformations, using homo-bifunctional crosslinkers such as the bifunctional imido esters having (CH₂)ₙ spacer groups with n=1 to n=6, glutaraldehyde, N-hydroxysuccinimide esters and hetero-bifunctional reagents which usually contain an amino-reactive moiety such as N-hydroxysuccinimide and another group specific-reactive moiety such as maleimido or dithio moiety (SH) or carbodiimide (COOH). In addition, peptides can be conformationally constrained by, for example, incorporation of C_{α} and N_{α}-methylamino acids, introduction of double bonds between C_{α} and C_{β} atoms of amino acids and the formation of cyclic peptides or analogs by introducing covalent bonds such as forming an amide bond between the N and C termini, between two side chains or between a side chain and the N or C terminus.

Accordingly, one aspect of the present invention contemplates any compound which binds or otherwise interacts with a TLR, such as TLR-2 or TLR-4, or a component of a TLR signaling pathway resulting in potentiation, activation or up-regulation or antagonism or down-regulation of the TLR or TLR signaling pathway.

Another useful group of compounds is a mimetic. The terms “peptide mimetic”, “target mimetic” or “mimetic” are intended to refer to a substance which has some chemical similarity to the target but which antagonizes or agonizes or mimics the target. The target in this case may be a TLR ligand. A peptide mimetic may be a peptide-containing molecule that mimics elements of protein secondary structure (Johnson et al., “Peptide Turn Mimetics” in Biotechnology and Pharmacy, Pezzuto et al., Eds., Chapman and Hall, New York, 1993). The underlying rationale behind the use of peptide mimetics is that the peptide backbone of proteins exists chiefly to orient amino acid side chains in such a way as to facilitate molecular interactions such as those of antibody and antigen, enzyme and substrate or scaffolding proteins. A peptide mimetic is designed to permit molecular interactions similar to the natural molecule. Peptide or non-peptide mimetics may be useful, for example, to activate a TLR or TLR pathway or to competitively inhibit a TLR. Preferred TLRs in this instance are TLR-2 and TLR-4.

Again, the compounds of the present invention may be selected to interact with a target alone or single or multiple compounds may be used to affect multiple targets. For example, multiple targets may include a TLR and the microorganism or virus itself.

The target TLR or fragment employed in screening assays may either be free in solution, affixed to a solid support, or borne on a cell surface. One method of drug screening utilizes eukaryotic or prokaryotic host cells which are stably transformed with recombinant polynucleotides expressing the TLR or fragment, preferably in competitive binding assays. Such cells, either in viable or fixed form, can be used for standard binding assays. One may measure, for example, the formation of complexes between a TLR or fragment and the agent being tested, or examine the degree to which the formation of a complex between a TLR or fragment and a ligand is aided or interfered with by the agent being tested.

A substance identified as a modulator of target function or gene activity may be a peptide or non-peptide in nature. Non-peptide “small molecules” are often preferred for many in vivo pharmaceutical uses. Accordingly, a mimetic or miiic of the substance (particularly if a peptide) may be designed for pharmaceutical use.

The designing of mimetics to a pharmaceutically active compound is a known approach to the development of pharmaceuticals based on a “lead” compound. This might be desirable where the active compound is difficult or expensive to synthesize or where it is unsuitable for a particular method of administration, e.g. peptides are unsuitable active agents for oral compositions as they tend to he quickly degraded by proteases in the alimentary canal. Mimetic design, synthesis and testing is generally used to avoid randomly screening large numbers of molecules for a target property.

There are several steps commonly taken in the design of a mimetic from a compound having a given target property. First, the particular parts of the compound that are critical and/or important in determining the target property are determined. In the case of a peptide, this can be done by systematically varying the amino acid residues in the peptide, e.g. by substituting each residue in turn. Alanine scans of peptides are commonly used to refine such peptide motifs. These parts or residues constituting the active region of the compound are known as its “pharmacophore”.

Once the pharmacophore has been found, its structure is modeled according to its physical properties, e.g. stereochemistry, bonding, size and/or charge, using data from a range of sources, e.g. spectroscopic techniques, x-ray diffraction data and NMR. Computational analysis, similarity mapping (which models the charge and/or volume of a pharmacophore, rather than the bonding between atoms) and other techniques can be used in this modeling process.

In a variant of this approach, the three-dimensional structure of a TLR and its ligand are modeled. This can be especially useful where the TLR and/or its ligand change conformation on binding, allowing the model to take account of this in the design of the mimetic. Modeling can be used to generate inhibitors which interact with the linear sequence or a three-dimensional configuration.

A template molecule is then selected onto which chemical groups which mimic the pharmacophore can be grafted. The template molecule and the chemical groups grafted onto it can conveniently be selected so that the mimetic is easy to synthesize, is likely to be pharmacologically acceptable, and does not degrade in vivo, while retaining the biological activity of the lead compound. Alternatively, where the mimetic is peptide-based, further stability can be achieved by cyclizing the peptide, increasing its rigidity. The mimetic or mimetics found by this approach can then be screened to see whether they have the target property, or to what extent they exhibit it. Further optimization or modification can then be carried out to arrive at one or more final mimetics for in vivo or clinical testing.

The goal of rational drug design is to produce structural analogs of biologically active polypeptides of interest or of small molecules with which they interact (e.g. agonists, antagonists, inhibitors or enhancers) in order to fashion drugs which are, for example, more active or stable forms of the polypeptide, or which, e.g. enhance or interfere with the function of a polypeptide in vivo. See, e.g. Hodgson (Bio/Technology 9: 19-21, 1991). In one approach, one first determines the three-dimensional structure of a TLR ligand by x-ray crystallography, by computer modeling or most typically, by a combination of approaches. Useful information regarding the structure of a TLR ligand may also be gained by modeling based on the structure of homologous proteins. An example of rational drug design is the development of HIV protease inhibitors (Erickson et al., Science 249: 527-533, 1990). In addition, target molecules may be analyzed by an alanine scan (Wells, Methods Enzymol. 202: 2699-2705, 1991). In this technique, an amino acid residue is replaced by Ala and its effect on the peptide's activity is determined. Each of the amino acid residues of the peptide is analyzed in this manner to determine the important regions of the peptide.

Proteomics may be also be used to screen for the differential production of components of the TLR signaling pathway or of particular TLRs such as TLR-2 and TLR-4 in response to different physiological conditions and/or in the presence of candidate drugs.

It is also possible to isolate a TLR-specific or TLR ligand-specific antibody (such as by the method described above) and then to solve its crystal structure. In principle, this approach yields a pharmacophore upon which subsequent drug design can be based. It is possible to bypass protein crystallography altogether by generating anti-idiotypic antibodies (anti-ids) to a functional, pharmacologically active antibody. As a mirror image of a mirror image, the binding site of the anti-ids would be expected to be an analog of the original receptor. The anti-id could then be used to identify and isolate peptides from banks of chemically or biologically produced banks of peptides. Selected peptides would then act as the pharmacophore.

The present invention extends to a genetic approach to up-regulating or down-regulating expression of a gene encoding a TLR, such as TLR-2 or TLR-4, or up- or down-regulating a compound in the TLR signaling pathway. Generally, it is more convenient to use genetic means to induce gene silencing such as pre- or post-transcriptional gene silencing. However, the general techniques can be used to up-regulate expression such as by increasing gene copy numbers or antagonizing inhibitors of gene expression.

The terms “nucleic acids”, “nucleotide” and “polynucleotide” include RNA, cDNA, genomic DNA, synthetic forms and mixed polymers, both sense and antisense strands, and may be chemically or biochemically modified or may contain non-natural or derivatized nucleotide bases, as will be readily appreciated by those skilled in the art. Such modifications include, for example, labels, methylation, substitution of one or more of the naturally occurring nucleotides with an analog (such as the morpholine ring), internucleotide modifications such as uncharged linkages (e.g. methyl phosphonates, phosphotriesters, phosphoamidates, carbamates, etc.), charged linkages (e.g. phosphorothioates, phosphorodithioates, etc.), pendent moieties (e.g. polypeptides), intercalators (e.g. acridine, psoralen, etc.), chelators, alkylators and modified linkages (e.g. α-anomeric nucleic acids, etc.). Also included are synthetic molecules that mimic polynucleotides in their ability to bind to a designated sequence via hydrogen binding and other chemical interactions. Such molecules are known in the art and include, for example, those in which peptide linkages substitute for phosphate linkages in the backbone of the molecule.

Antisense polynucleotide sequences, for example, are useful in silencing transcripts of TLR genes, such as TLR-2 or TLR-4 gene transcripts. Expression of such an antisense construct within a cell interferes with TLR gene transcription and/or translation. Furthermore, co-suppression and mechanisms to induce RNAi or siRNA may also be employed. Alternatively, antisense or sense molecules may be directly administered. In this latter embodiment, the antisense or sense molecules may be formulated in a composition and then administered by any number of means to target cells.

A variation on antisense and sense molecules involves the use of morpholinos, which are oligonucleotides composed of morpholine nucleotide derivatives and phosphorodiamidate linkages (for example, Summerton and Weller, Antisense and Nucleic Acid Drug Development 7: 187-195, 1997). Such compounds are injected into embryos and the effect of interference with mRNA is observed.

In one embodiment, the present invention employs compounds such as oligonucleotides and similar species for use in modulating the finction or effect of nucleic acid molecules such as those encoding a TLR, such as TLR-2 or TLR-4, i.e. the oligonucleotides induce pre-transcriptional or post-transcriptional gene silencing. This is accomplished by providing oligonucleotides which specifically hybridize with one or more nucleic acid molecules encoding the TLR gene transcription. The oligonucleotides may be provided directly to a cell or generated within the cell. As used herein, the terms “target nucleic acid” and “nucleic acid molecule encoding a TLR gene transcript” have been used for convenience to encompass DNA encoding the TLR, RNA (including pre-mRNA and mRNA or portions thereof) transcribed from such DNA, and also cDNA derived from such RNA. The hybridization of a compound of the subject invention with its target nucleic acid is generally referred to as “antisense”. Consequently, the preferred mechanism believed to be included in the practice of some preferred embodiments of the invention is referred to herein as “antisense inhibition.” Such antisense inhibition is typically based upon hydrogen bonding-based hybridization of oligonucleotide strands or segments such that at least one strand or segment is cleaved, degraded, or otherwise rendered inoperable. In this regard, it is presently preferred to target specific nucleic acid molecules and their functions for such antisense inhibition.

The functions of DNA to be interfered with can include replication and transcription. Replication and transcription, for example, can be from an endogenous cellular template, a vector, a plasmid construct or otherwise. The functions of RNA to be interfered with can include functions such as translocation of the RNA to a site of protein translation, translocation of the RNA to sites within the cell which are distant from the site of RNA synthesis, translation of protein from the RNA, splicing of the RNA to yield one or more RNA species, and catalytic activity or complex formation involving the RNA which may be engaged in or facilitated by the RNA. In one example, the result of such interference with TLR transcript function is reduced levels of the TLR. In the context of the present invention, “modulation” and “modulation of expression” mean either an increase (stimulation) or a decrease (inhibition) in the amount or levels of a nucleic acid molecule encoding the gene, e.g., DNA or RNA. Inhibition is often the preferred form of modulation of expression and MRNA is often a preferred target nucleic acid.

In the context of this invention, “hybridization” means the pairing of complementary strands of oligomeric compounds. In the present invention, the preferred mechanism of pairing involves hydrogen bonding, which may be Watson-Crick, Hoogsteen or reversed Hoogsteen hydrogen bonding, between complementary nucleoside or nucleotide bases (nucleobases) of the strands of oligomeric compounds. For example, adenine and thymine are complementary nucleobases which pair through the formation of hydrogen bonds. Hybridization can occur under varying circumstances.

An antisense compound is specifically hybridizable when binding of the compound to the target nucleic acid interferes with the normal function of the target nucleic acid to cause a loss of activity, and there is a sufficient degree of complementarity to avoid non-specific binding of the antisense compound to non-target nucleic acid sequences under conditions in which specific binding is desired, i.e. under physiological conditions in the case of in vivo assays or therapeutic treatment, and under conditions in which assays are performed in the case of in vitro assays.

“Complementary” as used herein, refers to the capacity for precise pairing between two nucleobases of an oligomeric compound. For example, if a nucleobase at a certain position of an oligonucleotide (an oligomeric compound), is capable of hydrogen bonding with a nucleobase at a certain position of a target nucleic acid, said target nucleic acid being a DNA, RNA, or oligonucleotide molecule, then the position of hydrogen bonding between the oligonucleotide and the target nucleic acid is considered to be a complementary position. The oligonucleotide and the further DNA, RNA, or oligonucleotide molecule are complementary to each other when a sufficient number of complementary positions in each molecule are occupied by nucleobases which can hydrogen bond with each other. Thus, “specifically hybridizable” and “complementary” are terms which are used to indicate a sufficient degree of precise pairing or complementarity over a sufficient number of nucleobases such that stable and specific binding occurs between the oligonucleotide and a target nucleic acid.

According to the present invention, compounds include antisense oligomeric compounds, antisense oligonucleotides, ribozymes, external guide sequence (EGS) oligonucleotides, alternate splicers, primers, probes, and other oligomeric compounds which hybridize to at least a portion of the target nucleic acid. As such, these compounds may be introduced in the form of single-stranded, double-stranded, circular or hairpin oligomeric compounds and may contain structural elements such as internal or terminal bulges or loops. Once introduced to a system, the compounds of the invention may elicit the action of one or more enzymes or structural proteins to effect modification of the target nucleic acid. One non-limiting example of such an enzyme is RNAse H, a cellular endonuclease which cleaves the RNA strand of an RNA:DNA duplex. It is known in the art that single-stranded antisense compounds which are “DNA-like” elicit RNAse H. Activation of RNase H, therefore, results in cleavage of the RNA target, thereby greatly enhancing the efficiency of oligonucleotide-mediated inhibition of gene expression. Similar roles have been postulated for other ribonucleases such as those in the RNase III and ribonuclease L family of enzymes.

While the preferred form of antisense compound is a single-stranded antisense oligonucleotide, in many species the introduction of double-stranded structures, such as double-stranded RNA (dsRNA) molecules, has been shown to induce potent and specific antisense-mediated reduction of the function of a gene or its associated gene products. This phenomenon occurs in both plants and animals.

In the context of the subject invention, the term “oligomeric compound” refers to a polymer or oligomer comprising a plurality of monomeric units. In the context of this invention, the term “oligonucleotide” refers to an oligomer or polymer of ribonucleic acid (RNA) or deoxyribonucleic acid (DNA) or mimetics, chimeras, analogs and homologs thereof. This term includes oligonucleotides composed of naturally occurring nucleobases, sugars and covalent internucleoside (backbone) linkages as well as oligonucleotides having non-naturally occurring portions which function similarly. Such modified or substituted oligonucleotides are often preferred over native forms because of desirable properties such as, for example, enhanced cellular uptake, enhanced affinity for a target nucleic acid and increased stability in the presence of nucleases.

While oligonucleotides are a preferred form of the compounds of this invention, the present invention comprehends other families of compounds as well, including but not limited to oligonucleotide analogs and mimetics such as those herein described.

The open reading frame (ORF) or “coding region” which is known in the art to refer to the region between the translation initiation codon and the translation termination codon, is a region which may be effectively targeted. Within the context of the present invention, one region is the intragenic region encompassing the translation initiation or termination codon of the open reading frame (ORF) of a gene.

Other target regions include the 5′ untranslated region (5′UTR), known in the art to refer to the portion of an mRNA in the 5′ direction from the translation initiation codon, and thus including nucleotides between the 5′ cap site and the translation initiation codon of an mRNA (or corresponding nucleotides on the gene), and the 3′ untranslated region (3′UTR), known in the art to refer to the portion of an mRNA in the 3′ direction from the translation termination codon, and thus including nucleotides between the translation termination codon and 3′ end of an mRNA (or corresponding nucleotides on the gene). The 5′ cap site of an mRNA comprises an N7-methylated guanosine residue joined to the 5′-most residue of the mRNA via a 5′-5′ triphosphate linkage. The 5′ cap region of an mRNA is considered to include the 5′ cap structure itself as well as the first 50 nucleotides adjacent to the cap site. It is also preferred to target the 5′ cap region.

Although some eukaryotic mRNA transcripts are directly translated, many contain one or more regions, known as “introns”, which are excised from a transcript before it is translated. The remaining (and, therefore, translated) regions are known as “exons” and are spliced together to form a continuous mRNA sequence. Targeting splice sites, i.e. intron-exon junctions or exon-intron junctions, may also be particularly useful in situations where aberrant splicing is implicated in disease, or where an overproduction of a particular splice product is implicated in disease. Aberrant fusion junctions due to rearrangements or deletions are also preferred target sites. mRNA transcripts produced via the process of splicing of two (or more) mRNAs from different gene sources are known as “fusion transcripts”. It is also known that introns can be effectively targeted using antisense compounds targeted to, for example, DNA or pre-mRNA.

As is known in the art, a nucleoside is a base-sugar combination. The base portion of the nucleoside is normally a heterocyclic base. The two most common classes of such heterocyclic bases are the purines and the pyrimidines. Nucleotides are nucleosides that further include a phosphate group covalently linked to the sugar portion of the nucleoside. For those nucleosides that include a pentofuranosyl sugar, the phosphate group can be linked to either the 2′, 3′ or 5′ hydroxyl moiety of the sugar. In forming oligonucleotides, the phosphate groups covalently link adjacent nucleosides to one another to form a linear polymeric compound. In turn, the respective ends of this linear polymeric compound can be further joined to form a circular compound, however, linear compounds are generally preferred. In addition, linear compounds may have internal nucleobase complementarity and may, therefore, fold in a manner as to produce a fully or partially double-stranded compound. Within oligonucleotides, the phosphate groups are commonly referred to as forming the internucleoside backbone of the oligonucleotide. The normal linkage or backbone of RNA and DNA is a 3′ to 5′ phosphodiester linkage.

Specific examples of preferred antisense compounds useful in this invention include oligonucleotides containing modified backbones or non-natural internucleoside linkages. As defined in this specification, oligonucleotides having modified backbones include those that retain a phosphorus atom in the backbone and those that do not have a phosphorus atom in the backbone. For the purposes of this specification, and as sometimes referenced in the art, modified oligonucleotides that do not have a phosphorus atom in their internucleoside backbone can also be considered to be oligonucleosides.

Preferred modified oligonucleotide backbones containing a phosphorus atom therein include, for example, phosphorothioates, chiral phosphorothioates, phosphorodithioates, phosphotriesters, aminoalkylphosphotriesters, methyl and other alkyl phosphonates including 3′-alkylene phosphonates, 5′-alkylene phosphonates and chiral phosphonates, phosphinates, phosphoramidates including 3′-amino phosphoramidate and aminoalkylphosphoramidates, thionophosphoramidates, thionoalkylphosphonates, thionoalkylphosphotriesters, selenophosphates and boranophosphates having normal 3′-5′ linkages, 2′-5′ linked analogs of these, and those having inverted polarity wherein one or more internucleotide linkages is a 3′ to 3′, 5′ to 5′ or 2′ to 2′ linkage. Preferred oligonucleotides having inverted polarity comprise a single 3′ to 3′ linkage at the 3′-most internucleotide linkage i.e. a single inverted nucleoside residue which may be abasic (the nucleobase is missing or has a hydroxyl group in place thereol). Various salts, mixed salts and free acid forms are also included.

The antisense oligonucleotides may be administered by any convenient means including by inhalation, local or systemic means.

In an alternative embodiment, genetic constructs including DNA vaccines are used to generate antisense molecules in vivo. Furthermore, many of the preferred features described above are appropriate for sense nucleic acid molecules or for gene therapy applications to promote levels of TLRs.

Following identification of an agent which potentiates or antagonizes a TLR or TLR pathway, it may be manufactured and/or used in a preparation, i.e. in the manufacture or formulation or a composition such as a medicament, pharmaceutical composition or drug. These may be administered to individuals in a method of treatment or prophylaxis of inection. Alternatively, they may be incorporated into a patch or slow release capsule or implant.

Thus, the present invention extends, therefore, to a pharmaceutical composition, medicament, drug or other composition including a patch or slow release formulation comprising an agonist or antagonist of TLR activity or TLR gene expression or the activity or gene expression of a component of the TLR signaling pathway.

Another aspect of the present invention contemplates a method comprising administration of such a composition to a subject such as for treatment or prophylaxis of an infection or other disease condition. Furthermore, the present invention contemplates a method of making a pharmaceutical composition comprising admixing a compound of the instant invention with a pharmaceutically acceptable excipient, vehicle or carrier, and optionally other ingredients. Where multiple compositions are provided, then such compositions may be given simultaneously or sequentially. Sequential administration includes administration within nanoseconds, seconds, minutes, hours or days. Preferably, sequential administration is within seconds or minutes.

Multi-part including two-art pharmaceutical compositions or packs are also contemplated comprising multiple components such as those which potentiate or inhibit a TLR such as TLR-2 or TLR4 together with anti-microbial or anti-viral agents. Such multi-part pharmaceutical compositions or packs maintain different agents or groups of agents separately. These are either dispensed separately or admixed prior to being dispensed.

Accordingly, another aspect of the present invention contemplates a method for the treatment or prophylaxis of an infection or other disease condition in a subject, said method comprising administering to said subject an effective amount of a compound as described herein or a composition comprising same.

Preferably, the subject is a mammal such as a human or laboratory test animal such as a mouse, rat, rabbit, guinea pig, hamster, zebrafish or amphibian or avian species such as a duck.

This method also includes providing a wild-type or mutant target gene function to a cell. This is particularly useful when generating an animal model. Alternatively, it may be part of a gene therapy approach. A target gene or a part of the gene may be introduced into the cell in a vector such that the gene remains extrachromosomal. In such a situation, the gene will be expressed by the cell from the extrachromosomal location. If a gene portion is introduced and expressed in a cell carrying a mutant target allele, the gene portion should encode a part of the target protein. Vectors for introduction of genes both for recombination and for extrachromosomal maintenance are known in the art and any suitable vector may be used. Methods for introducing DNA into cells such as electroporation calcium phosphate co-precipitation and viral transduction are known in the art.

Gene transfer systems known in the art may be useful in the practice of genetic manipulation. These include viral and non-viral transfer methods. A number of viruses have been used as gene transfer vectors or as the basis for preparing gene transfer vectors, including papovaviruses (e.g. SV40, Maczak et al., J. Gen. Virol. 73: 1533-1536, 1992), adenovirus (Berkner, Curr. Top. Microbiol. Immunol. 158: 39-66, 1992; Berkner et al., BioTechniques 6; 616-629, 1988; Gorziglia and Kapikian, J. Virol. 66: 4407-4412, 1992; Quantin et al., Proc. Natl. Acad. Sci. USA 89: 2581-2584, 1992; Rosenfeld et al., Cell 68: 143-155, 1992; Wilkinson et al., Nucleic Acids Res. 20: 2233-2239, 1992; Stratford-Perricaudet et al., Hum. Gene Ther. 1: 241-256, 1990; Schneider et al., Nature Genetics 18: 180-183, 1998), vaccinia virus (Moss, Curr. Top. Microbiol. Immunol. 158: 25-38, 1992; Moss, Proc. Natl. Acad. Sci. USA 93: 11341-11348, 1996), adeno-associated virus (Muzyczka, Curr. Top. Microbiol. Immunol. 158: 97-129, 1992; Ohi et al., Gene 89: 279-282, 1990; Russell and Hirata, Nature Genetics 18: 323-328, 1998), herpesviruses including HSV and EBV (Margolskee, Curr. Top., Microbiol. Immunol. 158: 67-95, 1992; Johnson et al., J. Virol. 66: 2952-2965, 1992; Fink et al., Hum. Gene Ther. 3: 11-19, 1992; Breakefield and Geller, Mol. Neurobiol. 1: 339-371, 1987; Freese et al., Biochem. Pharmacol. 40: 2189-2199, 1990; Fink et al., Ann. Rev. Neurosci. 19: 265-287, 1996), lentiviruses (Naldini et al., Science 272: 263-267, 1996), Sindbis and Semliki Forest virus (Berglund et al., Biotechnology 11: 916-920, 1993) and retroviruses of avian (Bandyopadhyay and Temin, Mol. Cell. Biol. 4: 749-754, 1984; Petropoulos et al., J. Viol. 66: 3391-3397, 1992], murine [Miller, Curr. Top. Microbiol. Immunol. 158: 1-24, 1992; Miller et al., Mol. Cell. Biol. 5: 431-437, 1985; Sorge et al., Mol. Cell. Biol. 4: 1730-1737, 1984; and Baltimore, J. Virol. 54: 401-407, 1985; Miller et al., J. Virol. 62: 4337-4345, 1988] and human [Shimada et al., J. Clin. Invest. 88: 1043-1047, 1991; Helseth et al., J. Virol. 64: 2416-2420, 1990; Page et al., J. Virol. 64: 5270-5276, 1990; Buchschacher and Panganiban, J. Virol. 66: 2731-2739, 1982] origin.

Non-viral gene transfer methods are known in the art such as chemical techniques including calcium phosphate co-precipitation, mechanical techniques, for example, microinjection, membrane fusion-mediated transfer via liposomes and direct DNA uptake and receptor-mediated DNA transfer. Viral-mediated gene transfer can be combined with direct in vivo gene transfer using liposome delivery, allowing one to direct the viralvectors to particular cells. Alternatively, the retroviral vector producer cell line can be injected into particular tissue. Injection of producer cells would then provide a continuous source of vector particles.

In an approach which combines biological and physical gene transfer methods, plasmid DNA of any size is combined with a polylysine-conjugated antibody specific to the adenovirus hexon protein and the resulting complex is bound to an adenovirus vector. The trimolecular complex is then used to infect cells. The adenovirus vector permits efficient binding, internalization and degradation of the endosome before the coupled DNA is damaged. For other techniques for the delivery of adenovirus based vectors, see U.S. Pat. No. 5,691,198.

Liposome/DNA complexes have been shown to be capable of mediating direct in vivo gene transfer. While in standard liposome preparations the gene transfer process is non-specific, localized in vivo uptake and expression have been reported in tumor deposits, for example, following direct in situ administration.

If the polynucleotide encodes a sense or antisense polynucleotide or a ribozyme or DNAzyme, expression will produce the sense or antisense polynucleotide or ribozyme or DNAzyme. Thus, in this context, expression does not require that a protein product be synthesized. In addition to the polynucleotide cloned into the expression vector, the vector also contains a promoter functional in eukaryotic cells. The cloned polynucleotide sequence is under control of this promoter. Suitable eukaryotic promoters include those described above. The expression vector may also include sequences, such as selectable markers and other sequences described herein.

Cells which carry mutant target alleles (e.g. TLR-2 or TLR-4) or where one or both alleles are deleted or up-regulated can be used as model systems to study the effects of infection or other disease condition.

The compounds, agents, medicaments, nucleic acid molecules and other target antagonists or agonists of the present invention can be formulated in pharmaceutical compositions which are prepared according to conventional pharmaceutical compounding techniques. See, for example, Remington's Pharmaceutical Sciences, 18^{th} Ed. (1990, Mack Publishing, Company, Easton, Pa., U.S.A.). The composition may contain the active agent or pharmaceutically acceptable salts of the active agent. These compositions may comprise, in addition to one of the active substances, a pharmaceutically acceptable excipient, carrier, buffer, stabilizer or other materials well known in the art. Such materials should be non-toxic and should not interfere with the efficacy of the active ingredient. The carrier may take a wide variety of forms depending on the form of preparation desired for administration, e.g. topical, intravenous, oral, intrathecal, epineural or parenteral.

For oral administration, the compounds can be formulated into solid or liquid preparations such as capsules, pills, tablets, lozenges, powders, suspensions or emulsions. In preparing the compositions in oral dosage form, any of the usual pharmaceutical media may be employed, such as, for example, water, glycols, oils, alcohols, flavoring agents, preservatives, coloring agents, suspending agents, and the like in the case of oral liquid preparations (such as, for example, suspensions, elixirs and solutions); or carriers such as starches, sugars, diluents, granulating agents, lubricants, binders, disintegrating agents and the like in the case of oral solid preparations (such as, for example, powders, capsules and tablets). Because of their ease in administration, tablets and capsules represent the most advantageous oral dosage unit form, in which case solid pharmaceutical carriers are obviously employed. If desired, tablets may be sugar-coated or enteric-coated by standard techniques. The active agent can be encapsulated to make it stable to passage through the gastrointestinal tract while at the same time allowing for passage across the blood brain barrier. See for example, International Patent Publication No. WO 96/11698.

For parenteral administration, the compound may dissolved in a pharmaceutical carrier and administered as either a solution of a suspension. Illustrative of suitable carriers are water, saline, dextrose solutions, fructose solutions, ethanol, or oils of animal, vegetative or synthetic origin. The carrier may also contain other ingredients, for example, preservatives, suspending agents, solubilizing agents, buffers and the like. When the compounds are being administered intrathecally, they may also be dissolved in cerebrospinal fluid.

The active agent is preferably administered in a therapeutically effective amount. The actual amount administered and the rate and time-course of administration will depend on the nature and severity of the condition being treated. Prescription of treatment, e.g. decisions on dosage, timing, etc. is within the responsibility of general practitioners or specialists and typically takes account of the disorder to be treated, the condition of the individual patient, the site of delivery, the method of administration and other factors known to practitioners. Examples of techniques and protocols can be found in Remington's Pharmaceutical Sciences, supra.

Alternatively, targeting therapies may be used to deliver the active agent more specifically to certain types of cell, by the use of targeting systems such as antibodies or cell specific ligands or specific nucleic acid molecules. Targeting may be desirable for a variety of reasons, e.g. if the agent is unacceptably toxic or if it would otherwise require too high a dosage or if it would not otherwise be able to enter the target cells.

Instead of administering these agents directly, they could be produced in the target cell, e.g. in a viral vector such as described above or in a cell based delivery system such as described in U.S. Pat. No. 5,550,050 and International Patent Publication Nos. WO 92/19195, WO 94/25503, WO 95/01203, WO 95/05452, WO 96/02286, WO 96/02646, WO 96/40871, WO 96/40959 and WO 97/12635. The vector could be targeted to the target cells. The cell based delivery system is designed to be implanted in a patient's body at the desired target site and contains a coding sequence for the target agent. Alternatively, the agent could be administered in a precursor form for conversion to the active form by an activating agent produced in, or targeted to, the cells to be treated. See, for example, European Patent Application No. 0 425 731A and International Patent Publication No. WO 90/07936.

The present invention further contemplates diagnostic protocols such as to determine the presence or absence of infection or other disease condition, whether an infection has become chronic, the susceptibility of a subject to infection and/or the efficacy of a therapeutic protocol.

Immunological based TLR detection protocols may take a variety of forms. For example, a plurality of antibodies may be immobilized in an array each with different specificities to particular TLRs or monocytes or hepatocytes comprising TLRs. Cells from a biopsy are then brought into contact with the antibody array and a diagnosis may be made as to the level and type of TLRs elevated or down-regulated on the cell.

Other more conventional assays may also be conducted such as by ELISA, Western blot analysis, immunoprecipitation analysis, immunofluorescence analysis, immunochemistry analysis or FACS analysis.

The present invention provides, therefore, a method of detecting in a TLR or cell comprising same or fragment, variant or derivative thereof comprising contacting the sample with an antibody or fragment or derivative thereof and detecting the level of a complex comprising said antibody and the TLR or fragment, variant or derivative thereof compared to normal controls wherein altered levels of the TLR is indicative of the presence or absence of infection or other disease condition.

Preferably, the TLR is TLR-2 and/or TLR-4. Preferably, the other disease condition is liver disease such as cirrhosis or HCC.

As discussed above, any suitable technique for determining formation of the complex may be used. For example, an antibody according to the invention, having a reporter molecule associated therewith, may be utilized in immunoassays. Such immunoassays include but are not limited to radioimmunoassays (RIAs), enzyme-linked immunosorbent assays (ELISAs) and immunochromatographic techniques (ICTs), Western blotting which are well known to those of skill in the art. For example, reference may be made to Coligan et al., 1991-1997, supra which discloses a variety of immunoassays which may be used in accordance with the present invention. Immunoassays may include competitive assays. It will be understood that the present invention encompasses qualitative and quantitative immunoassays.

Suitable immunoassay techniques are described, for example, in U.S. Pat. Nos. 4,016,043, 4,424,279 and 4,018,653. These include both single-site and two-site assays of the non-competitive types, as well as the traditional competitive binding assays. These assays also include direct binding of a labeled antigen-binding molecule to a target antigen. The antigen in this case is the TLR or a fragment thereof.

Two-site assays are particularly favoured for use in the present invention. A number of variations of these assays exist, all of which are intended to be encompassed by the present invention. Briefly, in a typical forward assay, an unlabeled antigen-binding molecule such as an unlabeled antibody is immobilized on a solid substrate and the sample to be tested brought into contact with the bound molecule. After a suitable period of incubation, for a period of time sufficient to allow formation of an antibody-antigen complex, another antigen-binding molecule, suitably a second antibody specific to the antigen, labeled with a reporter molecule capable of producing a detectable signal is then added and incubated, allowing time sufficient for the formation of another complex of antibody-antigen-labeled antibody. Any unreacted material is washed away and the presence of the antigen is determined by observation of a signal produced by the reporter molecule. The results may be either qualitative, by simple observation of the visible signal, or may be quantitated by comparing with a control sample containing known amounts of antigen. Variations on the forward assay include a simultaneous assay, in which both sample and labeled antibody are added simultaneously to the bound antibody. These techniques are well known to those skilled in the art, including minor variations as will be readily apparent.

In the typical forward assay, a first antibody having specificity for the antigen or antigenic parts thereof is either covalently or passively bound to a solid surface. The solid surface is typically glass or a polymer, the most commonly used polymers being cellulose, polyacrylamide, nylon, polystyrene, polyvinyl chloride or polypropylene. The solid supports may be in the form of tubes, beads, discs of microplates, or any other surface suitable for conducting an immunoassay. The binding processes are well known in the art and generally consist of cross-linking covalently binding or physically adsorbing, the polymer-antibody complex is washed in preparation for the test sample. An aliquot of the sample to be tested is then added to the solid phase complex and incubated for a period of time sufficient and under suitable conditions to allow binding of any antigen present to the antibody. Following the incubation period, the antigen-antibody complex is washed and dried and incubated with a second antibody specific for a portion of the antigen. The second antibody has generally a reporter molecule associated therewith that is used to indicate the binding of the second antibody to the antigen. The amount of labeled antibody that binds, as determined by the associated reporter molecule, is proportional to the amount of antigen bound to the immobilized first antibody.

An alternative method involves immobilizing the antigen in the biological sample and then exposing the immobilized antigen to specific antibody that may or may not be labeled with a reporter molecule. Depending on the amount of target and the strength of the reporter molecule signal, a bound antigen may be detectable by direct labelling with the antibody. Alternatively, a second labeled antibody, specific to the first antibody is exposed to the target-first antibody complex to form a target-first antibody-second antibody tertiary complex. The complex is detected by the signal emitted by the reporter molecule.

From the foregoing, it will be appreciated that the reporter molecule associated with the antigen-binding molecule may include the following:
(a) direct attachment of the reporter molecule to the antibody;
(b) indirect attachment of the reporter molecule to the antibody; i.e., attachment of the reporter molecule to another assay reagent which subsequently binds to the antibody; and
(c) attachment to a subsequent reaction product of the antibody.

The reporter molecule may be selected from a group including a chromogen, a catalyst, an enzyme, a fluorochrome, a chemiluminescent molecule, a paramagnetic ion, a lanthanide ion such as Europium (Eu³⁴), a radioisotope including other nuclear tags and a direct visual label.

In the case of a direct visual label, use may be made of a colloidal metallic or non-metallic particle, a dye particle, an enzyme or a substrate, an organic polymer, a latex particle, a liposome, or other vesicle containing a signal producing substance and the like.

A large number of enzymes suitable for use as reporter molecules is disclosed in U.S. Pat. No. 4,366,241, U.S. Pat. No. 4,843,000, and U.S. Pat. No. 4,849,338. Suitable enzymes useful in the present invention include alkaline phosphatase, horseradish peroxidase, luciferase, β-galactosidase, glucose oxidase, lysozyme, malate dehydrogenase and the like. The enzymes may be used alone or in combination with a second enzyme that is in solution.

Suitable fluorochromes include, but are not limited to, fluorescein isothiocyanate (FITC), tetramethylrhodamine isothiocyanate (TRITC), R-Phycoerythrin (RPE), and Texas Red.

Other exemplary fluorochromes include those discussed by International Patent Publication No. WO 93/06121. Reference also may be made to the fluorochromes described in U.S. Pat. Nos. 5,573,909 and 5,326,692. Alternatively, reference may be made to the fluorochromes described in U.S. Pat. Nos. 5,227,487, 5,274,113, 5,405,975, 5,433,896, 5,442,045, 5,451,663, 5,453,517, 5,459,276, 5,516,864, 5,648,270 and 5,723,218.

In the case of an enzyme immunoassay, an enzyme is conjugated to the second antibody, generally by means of glutaraldehyde or periodate. As will be readily recognized, however, a wide variety of different conjugation techniques exist which are readily available to the skilled artisan. The substrates to be used with the specific enzymes are generally chosen for the production of, upon hydrolysis by the corresponding enzyme, a detectable colour change. Examples of suitable enzymes include those described supra. It is also possible to employ fluorogenic substrates, which yield a fluorescent product rather than the chromogenic substrates noted above. In all cases, the enzyme-labeled antibody is added to the first antibody-antigen complex, allowed to bind, and then the excess reagent washed away. A solution containing the appropriate substrate is then added to the complex of antibody-antigen-antibody. The substrate will react with the enzyme linked to the second antibody, giving a qualitative visual signal, which may be further quantitated, usually spectrophotometrically, to give an indication of the amount of antigen which was present in the sample.

Alternately, fluorescent compounds, such as fluorescein, rhodamine and the lanthanide, europium (EU), may be chemically coupled to antibodies without altering their binding capacity. When activated by illumination with light of a particular wavelength, the fluorochrome-labeled antibody adsorbs the light energy, inducing a state to excitability in the molecule, followed by emission of the light at a characteristic colour visually detectable with a light microscope. The fluorescent-labeled antibody is allowed to bind to the first antibody-antigen complex. After washing off the unbound reagent, the remaining tertiary complex is then exposed to light of an appropriate wavelength. The fluorescence observed indicates the presence of the antigen of interest. Immunofluorometric assays (IFMA) are well established in the art and are particularly useful for the present method. However, other reporter molecules, such as radioisotope, chemiluminescent or bioluminescent molecules may also be employed.

Monoclonal antibodies to a TLR may also be used in ELISA-mediated detection of the TLR. This may be undertaken in any number of ways such as immobilizing anti-TLR antibodies to a solid support and contacting these with PBMCs and/or liver cells. Labeled anti-TLR antibodies are then used to detect immobilized TLR. Alternatively, antibodies to other PBMC or liver cell surface markers are used. This assay may be varied in any number of ways and all variations are encompassed by the present invention. This approach enables rapid detection and quantitation of TLR levels.

The subject antibodies are also useful in in situ hybridization analysis such as of biopsy material. Such analysis enables the rapid diagnosis of levels of TLRs such as TLR-2 and TLR-4.

In another embodiment, the method for detection comprises detecting the level of expression in a cell of a polynucleotide encoding a TLR. Expression of such a polynucleotide may be determined using any suitable technique. For example, a labeled polynucleotide encoding a TLR may be utilized as a probe in a Northern blot of an RNA extract obtained from the cell. Preferably, a nucleic acid extract from the animal is utilized in concert with oligonucleotide primers corresponding to sense and antisense sequences of a polynucleotide encoding the kinase, or flanking sequences thereof, in a nucleic acid amplification reaction such as RT PCR. A variety of automated solid-phase detection techniques are also appropriate. For example, a very large scale immobilized primer arrays (VLSIPS (trademark)) are used for the detection of nucleic acids as, for example, described by Fodor et al. (Science 251: 767-777, 1991) and Kazal et al. (Nature Medicine 2: 753-759, 1996). The above genetic techniques are well known to persons skilled in the art.

For example, to a TLR encoding RNA transcript, RNA is isolated from a cellular sample suspected of containing TLR RNA, e.g. total RNA isolated from human PBMCs. RNA can be isolated by methods known in the art, e.g. using TRIZOL (trademark) reagent (GIBCO-BRL/Life Technologies, Gaithersburg, Md.). Oligo-dT, or random-sequence oligonucleotides, as well as seuqence-specific oligonucleotides can be employed as a primer in a reverse transcriptase reaction to prepare first-strand cDNAs from the isolated RNA. Resultant first-strand cDNAs are then amplified with sequence-specific oligonucleotides in PCR reactions to yield an amplified product.

“Polymerase chain reaction” or “PCR” refers to a procedure or technique in which amounts of a preselected fragment of nucleic acid, RNA and/or DNA, are amplified as described in U.S. Pat. No. 4,683,195. Generally, sequence information from the ends of the region of interest or beyond is employed to design oligonucleotide primers. These primers will be identical or similar in sequence to opposite strands of the template to be amplified. PCR can be used to amplify specific RNA sequences and cDNA transcribed from total cellular RNA. See generally Mullis et al. (Quant. Biol. 51: 263, 1987; Erlich, eds., PCR Technology, Stockton Press, NY, 1989). Thus, amplification of specific nucleic acid sequences by PCR relies upon oligonucleotides or “primers” having conserved nucleotide sequences wherein the conserved sequences are deduced from alignments of related gene or protein sequences, e.g. a sequence comparison of mammalian TLR genes. For example, one primer is prepared which is predicted to anneal to the antisense strand and another primer prepared which is predicted to anneal to the sense strand of a CDNA molecule which encodes a TLR.

To detect the amplified product, the reaction mixture is typically subjected to agarose gel electrophoresis or other convenient separation technique and the relative presence of the TLR specific amplified DNA detected. For example, TLR amplified DNA may be detected using Southern hybridization with a specific oligonucleotide probe or comparing is electrophoretic mobility with DNA standards of known molecular weight. Isolation, purification and characterization of the amplified TLR DNA may be accomplished by excising or eluting the fragment from the gel (for example, see references Lawn et al., Nucleic Acids Res. 2: 6103, 1981; Goeddel et al., Nucleic cids Res. 8: 4057-1980), cloning the amplified product into a cloning site of a suitable vector, such as the pCRII vector (Invitrogen), sequencing the cloned insert and comparing the DNA sequence to the known sequence of TLR. The relative amounts of TLR MRNA and cDNA can then be determined.

Real-time PCR is particularly useful in determining transcriptional levels of PCR genes. Determination of transcriptional activity also includes a measure of potential translational activity based on available mRNA transcripts. Real-time PCR as well as other PCR procedures use a number of chemistries for detection of PCR product including the binding of DNA binding fluorophores, the 5′ endonuclease, adjacent liner and hairpin oligoprobes and the self-fluorescing amplicons. These chemistries and real-time PCR in general are discussed, for example, in Mackay et al., Nucleic Acids Res 30(6): 1292-1305, 2002; Walker, J. Biochem. Mol. Toxicology 15(3): 121-127, 2001; Lewis et al., J. Pathol. 195: 66-71, 2001.

The present invention further provides gene arrays and/or gene chips to screen for the up- or down-regulation of mRNA transcripts. This aspect of the present invention is particularly useful in identifying conditions which result in the up- or down-regulation of TLR gene transcripts. Furthermore, compounds can be readily screened which up- or down-regulate TLR transcripts and in particular TLR-2 and TLR-4 transcripts.

The present invention is further described by the following non-limiting Examples.

### EXAMPLE 1

### Effects of HCV on TLR-2 and TLR-4 Expression

Patients

The study group included 16 outpatients attending a specialist Liver Clinic at a university teaching hospital with biopsy proven chronic Hepatitis C (CHC) [Tables 3 and 4]. Thirty-two age- and sex-matched, asymptomatic volunteers with no history of liver disease, alcohol intake <20 g/day and normal liver function tests served as controls. Ethical approval was obtained from the South Eastern Area Health Service Research Ethics Committee, Department of Health, New South Wales, Australia.

Blood Sampling

Peripheral blood was drawn using pyrogen-free needles, syringes and containers (Becton-Dickinson, Singapore). Plasma and serum were separated in a refrigerated centrifuge at 4° C. and stored at −70° C. in pyrogen-free polyethylene cryotubes (Nunc, Denmark) until analysis within six weeks of collection. Whole blood was used for determination of TLR expression on peripheral blood monocytes.

Serum TNF-α Assay

Serum TNF-α was measured using the Quantikine HS Human TNF-γ Immunoassay (R&D Systems Inc., Minneapolis, USA), according to the manufacturer's instructions. Sensitivity of the assay was 0.5 pg/mL.

Flow Cytometry for Determination of TLR Expression

Cell surface staining was performed on whole blood using the following anti-human monoclonal antibodies: anti-TLR-2-fluoroisothiocyanate (FITC) and anti-TLR-4-phycoerythrin (PE) (eBioscience) and anti-CD14-peridinin chlorophyll protein (PerCP) (Becton Dickinson, USA). Isotype matched non-binding controls were used for comparison. Ten thousand CD14-positive cells were acquired for each sample and dead cells were gated out based on their light scatter properties on FACSCaliber flow cytometer (Becton Dickinson, USA). At least two control patients were used for standardization purposes every flow cytometry session. TLR-2 and TLR-4 values were expressed as a ratio of the geometric mean fluorescence of individual study patients to mean control values for that session.

TABLE 3
Patient Characterisitics

| | Patients | Controls |
|---|---|---|
| Number of patients | 16 | 32 |
| Mean Age ± sd | 38.89 ± 8.67 | 35.44 ± 8.11 |
| Sex (M:F) | 10:6 | 20:12 |

TABLE 4
Characteristics of the CHC patients

Characteristic

| | |
|---|---|
| Genotype | 1: n − 11, 2: n = 2, 3: n = 3 |
| Ishak histological | Median: 4 range 2-7 |
| activity score | |
| ALT | Median 109 range 55-350 U/L (N: 15-45 U/L) |

This Example demonstrates that CD14^{+ve} monocyte expression of TLR-2 and of TLR-4 is significantly up-regulated in HCV patients and that the TLR-2 level correlates significantly with circulating TNF-α ALT levels. These findings indicate that the up-regulation in the immune receptors may be a direct effect of the HCV. In addition cell signaling via TLR-2, but not TLR-4, likely contributes to the increased circulating levels of TNF-α and the corresponding increased ALT levels found in CHC. The good correlation between TNF-α and ALT levels may indicate a direct causative mechanism for some of the hepatic destruction in HCV infection.

### EXAMPLE 2

### Effect of HBV Infection on TLR-2 Expression

Eighteen non-cirrhotic patients with chronic Hepatitis B (CHB) and on-going viral replication (HBV DNA >200,000 genomes/mL, n=12 and 200-10,000 genomes/mL, n=6; Cobas Amplicor HBV Monitor (trademark) Test, USA) and 32 healthy control subjects were studied. TLR-2 and TLR-4 expression on CD14^{+ve} peripheral blood mononuclear cells (PBMC's) was measured by flow cytometry using anti-CD14 (Becton Dickinson) and anti-TLR-2 and anti-TLR-4 (eBioscience, USA) monoclonal antibodies. TLR expression was reassessed in five patients in whom HBV DNA fell from >200,000 to <200 genomes/mL following treatment with lamivudine. In vitro TLR-2 expression by PBMCs was measured in five control subjects at baseline and following stimulation for 20 h by partially purified recombinant HBV. TLR-2 expression (expressed as a ratio to control results) was significantly reduced in chronic hepatitis B patients with HBV DNA >200,000 genomes/mL (median: 0.63; range: 0.05-1.52) compared with controls (P=0.001) and those with HBV DNA 200-10,000 genomes/mL (median: 0.98; range: 0.94-1.17) (P=0.04). TLR-4 expression did not differ significantly between the three groups. TLR-2 expression normalised in each of the five lamivudine-treated CHB patients in whom HBV DNA became undetectable. In vitro expression of TLR-2 fell in a concentration-dependent manner following exposure to recombinant HBV.

This Example shows that HBV down-regulates expression of TLR-2 on PBMC's. It is proposed herein that the HBV-induced defect in innate immunity contributes to the development of persistent infection.

### EXAMPLE 3

### Expression of TLRs in Cirrhosis

Patients

The study group included 36 outpatients attending a specialist Liver Clinic at a university teaching hospital with cirrhosis due to a range of aetiologies and covering the spectrum of degrees of hepatic functional impairment as reflected by the Child-Pugh classification (Pugh et al., Br J Surg. 60: 646-649, 1973) (Table 5). Eight patients were receiving treatment for hepatic encephalopathy with lactulose (β-galactofructosidase; Solvay Pharmaceuticals, Sydney, Australia), of relevance as this non-absorbable disaccharide reduces the intestinal content of endotoxin-containing Gram-negative gut flora (van Leeuwen et al., Surgery 110: 169-174, 1991). Patients were considered to have alcohol-related cirrhosis if alcohol intake had been in excess of 80 g/day in males and 30 g/day in females for more than five years and if testing for viral, metabolic and immune aetiologies was negative (Hanck et al., 2001, supra). Only patients who had been abstinent from alcohol for at least three months were included, as alcohol influences the sensitivity of macrophages to endotoxin and the production of TNF-α by PBMCs (Manrekar et al., Alcohol Clin Exp Res. 21: 1226-1231, 1997; Enomoto et al., Gastroenterology 115: 443-451, 1998). Patients with histological features of alcoholic hepatitis were excluded. Exclusion criteria also included a history within the previous six weeks of other factors which may influence circulating endotoxin and/or INF-α concentrations, such as infection, antibiotic or immunomodulatory drug use or gastrointestinal hemorrhage. As TNF-α is cleared by the kidney, patients with renal insufficiency (serum creatinine >20 μmol/L) were also excluded (von Baehr et al., 2000, supra; Hanck et al., 2001, supra; Bigatello et al., Am J Gastroenterol. 82: 11-15, 1987). Thirty-two age- and sex-matched, asymptomatic volunteers with no history of liver disease, alcohol intake <20 g/day and normal liver function tests served as controls. Informed consent in writing was obtained from each patient. The study protocol conformed to the ethical guidelines of the 1975 Declaration of Helsinki as reflected in a priori approval by the South Eastern Area Health Service Research Ethics Committee, Department of Health, New South Wales, Australia.

Blood Sampling

Peripheral blood was drawn using pyrogen-free needles, syringes and containers (Becton-Dickinson, Singapore). Plasma and serum were separated in a refrigerated centrifuge at 4° C. and stored at −70° C. in pyrogen-free polyethylene cryotubes (Nunc, Denmark) until analysis within six weeks of collection. Whole blood was used for determination of TLR expression on peripheral blood monocytes.

Plasma Endotoxin Assay

Plasma endotoxin was measured using the chromogenic limulus amoebocyte lysate assay (Associate of Cape Cod Inc., MA, USA), according to the manufacturer's instructions. Sensitivity of the assay was 3 pg/mL.

Serum TNF-α Assay

Serum TNF-α was measured using the Quantikine HS Human TNF-α Immunoassay (R&D Systems Inc., Minneapolis, USA), according to the manufacturer's instructions. Sensitivity of the assay was 0.5 pg/mL.

Serum sTNFR Assays

Serum sTNF RI (p55) and sTNF RII (p75) were measured using Quantikine HS Human sTNFR Immunoassays (R&D Systems Inc., Minneapolis, USA), according to the manufacturer's instructions. Sensitivity of the sTNF RI and sTNF RII assays were 3.0 pg/mL and 1.0 pg/mL, respectively.

Flow Cytometry for Determinatiion of TLR Expression

Cell surface staining was performed on whole blood using the following anti-human monoclonal antibodies: anti-TLR-2-fluoroisothiocyanate (FITC) and anti-TLR-4-phycoerythrin (PE) (eBioscience) and anti-CD14-peridinin chlorophyll protein (PerCP) (Becton Dickinson, USA). Isotype matched non-binding controls were used for comparison. Ten thousand CD14^{+ve} cells were acquired for each sample and dead cells were gated out based on their light scatter properties on FACSCaliber flow cytometer (Becton Dickinson, USA). At least two control patients were used for standardization purposes every flow cytometry session. TLR-2 and TLR-4 values were expressed as a ratio of the geometric mean fluorescence of individual study patients to mean control values for that session.

In Vitro Production of TNF-α by PBMCs PBMC's were isolated from whole blood on a Ficoll-Paque gradient (Pharmacia; Uppsala, Sweden). After washing, PBMCs were adjusted to 2×10⁶ cells per ml in RPMI supplemented with antibiotics and 3% v/v heat inactivated fetal bovine serum. PBMCs were plated in 96-well round-bottom tissue culture plates at 2×10⁵ cells per 200 μl media. Cells were stimulated in duplicate with endotoxin or staphylococcus enterotoxin B (SEB), each at concentrations of 10 ng/mL. These concentrations were shown in preliminary experiments to produce consistent TNF-α responses in control subjects. Supernatants were harvested after 20 h and stored at −20° C. until analysis. TNF-α was measured by capture ELISA (TNF-α OptEIA (trademark), BD Pharmingen, California, USA) according to the manufacturer's specifications with only minor modifications. ELISA sensitivity was 8 pg/mL. Values were expressed as the ratio of the TNF-α concentration in the culture supernatant of endotoxin- or SEB-stimulated cells to that of unstimulated cells for each individual control and cirrhotic patient.

Oral Supplementation with a Synbiotic Gram-Positive Gut Flora Regimen

Eleven patients with cirrhosis (alcohol=7; hepatitis C virus=3 and primary biliary cirrhosis=1; Child-Pugh class A=7; class B=2; class C=2) received oral supplementation with a freeze-dried Gram-positive gut flora regimen including Lactobacillus plantarum 2362, Lactobacillus paracasei subsp paracasei 19, Pediacoccus pentoseceus 5-33:3 and Lactococcus raffinolactis 32-77:1, each at a concentration of 4×10¹⁰ colony forming units per sachet, along with 10 g of bioactive fibre (betaglucan, 2.5 g; inulin, 2.5 g; pectin, 2.5 g; resistant starch, 2.5 g) (Synbiotic 2000; Medipharm, Kagerod, Sweden). Patients received one sachet twice daily for seven days. No patient was treated with lactulose prior to or during the period of intervention. TLR-2 expression on PBMC's, serum TNF-α levels and in vitro production of TNF-α by PBMCs in response to stimulation with SEB were determined at baseline, after seven days of supplementation and 28 days post-cessation of supplementation. Five asymptomatic volunteers, not supplemented with the gut flora regimen, served as controls for the purpose of standardization of TLR-2 analyses at these three time points.

Statistical Analyses

Statistical analyses were performed using the Kruskal-Wallis test, the Mann-Whitney rank sum test, Spearman's rank correlation test and the Wilcoxon rank sum test, as appropriate (Systat for Windows, version 5.02, Systat Inc., Evanston, Ill., USA). The probability level of P≦0.05 was set for statistical significance.

Plasma Endotoxin Levels

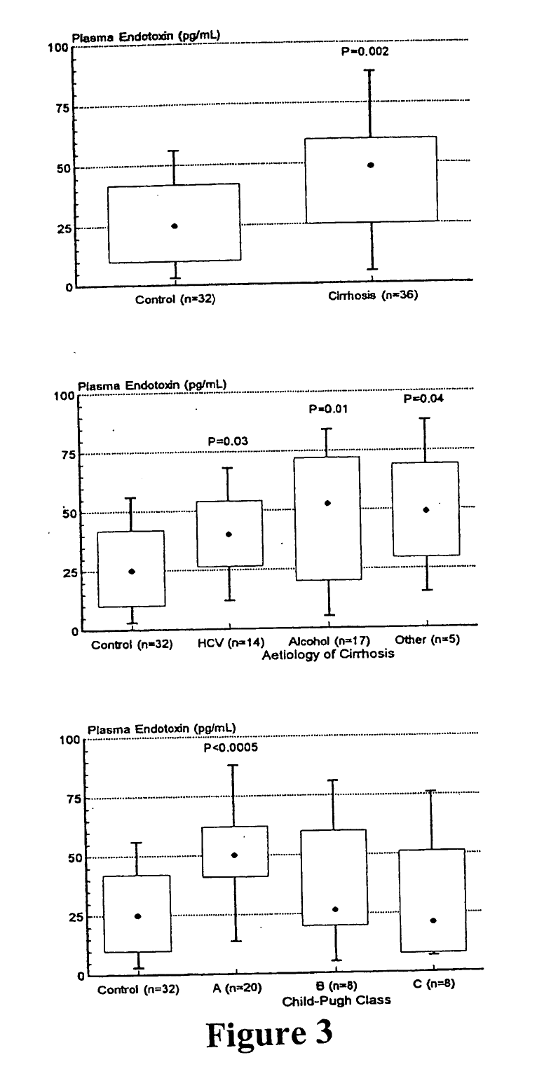

Plasma endotoxin levels were significantly increased in the cirrhotic patients compared to healthy controls, irrespective of aetiology (FIG. 3). When examined in relation to Child-Pugh grade, elevated endotoxin levels were found in patients with grade A cirrhosis but not in those with more advanced degrees of hepatic dysfunction (grades B and C) (FIG. 3).

Serum INF-α Levels

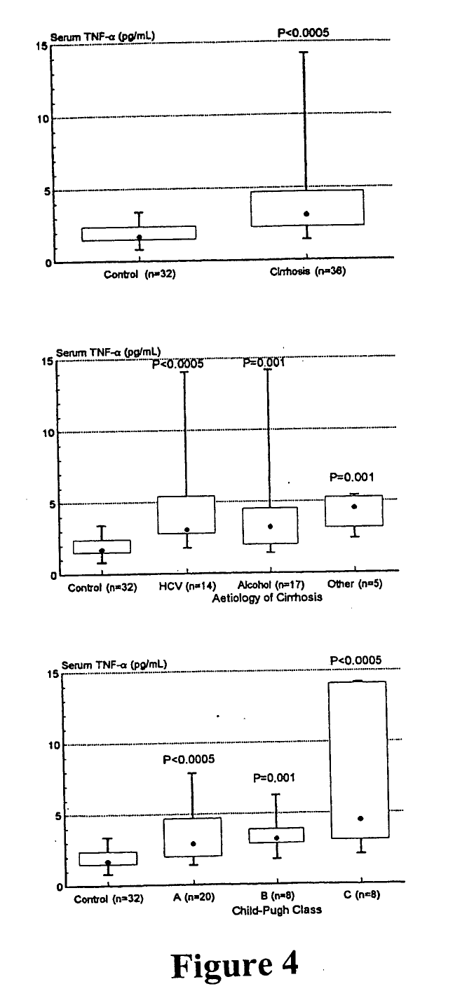

Serum TNF-α levels were significantly higher in cirrhotic patients than control subjects, irrespective of the aetiology of cirrhosis or Child-Pugh grade (FIG. 4). Levels were not significantly correlated with plasma endotoxin levels (r=0.01, P=0.99).

Serum sTNFR Levels

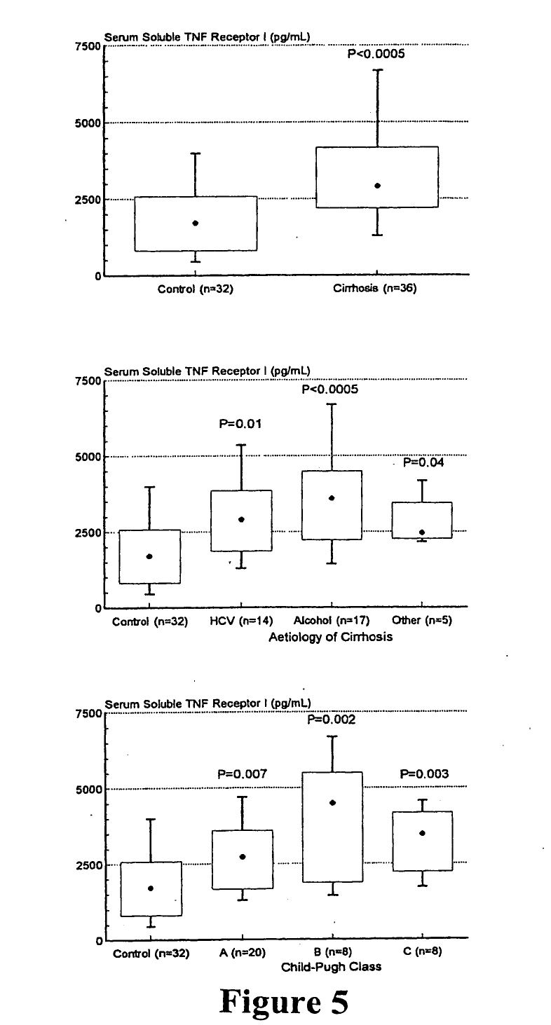

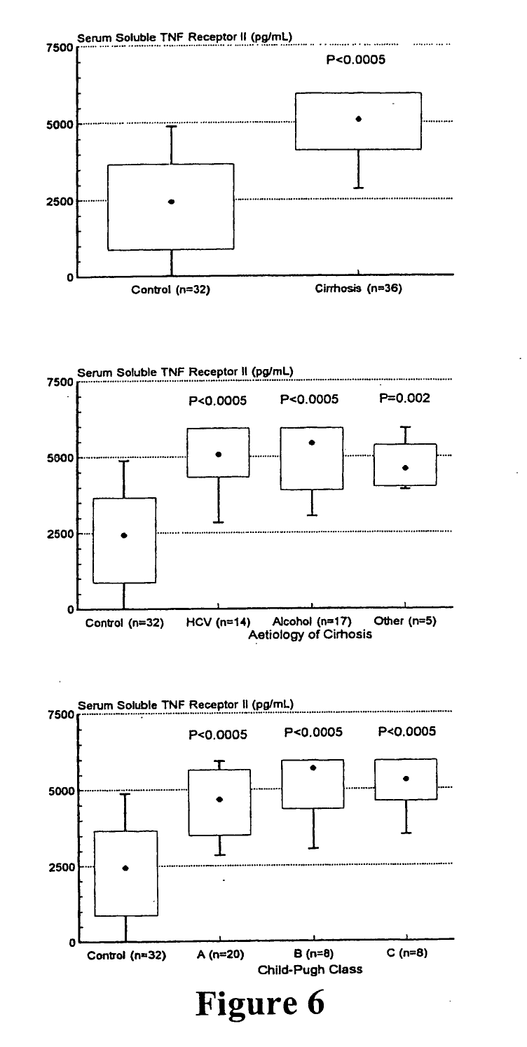

Serum sTNF RI and sTNF RII levels were each significantly higher in cirrhotic patients than controls, irrespective of aetiology of cirrhosis or Child-Pugh grade (FIGS. 5 and 6). sTNF RI and sTNF RII levels were not significantly correlated with plasma endotoxin levels (r=0.07, P=0.80 and r=0.21, P=0.09, respectively).

TLR-4 and TLR-2 Expression on CD14^{+ve} Peripheral Blood Monocytes (Gated from Whole Blood)

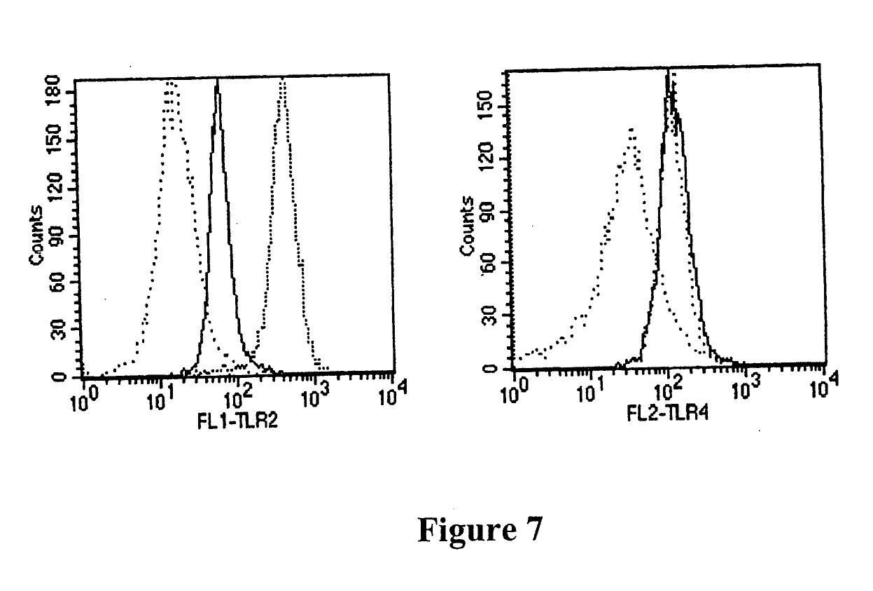

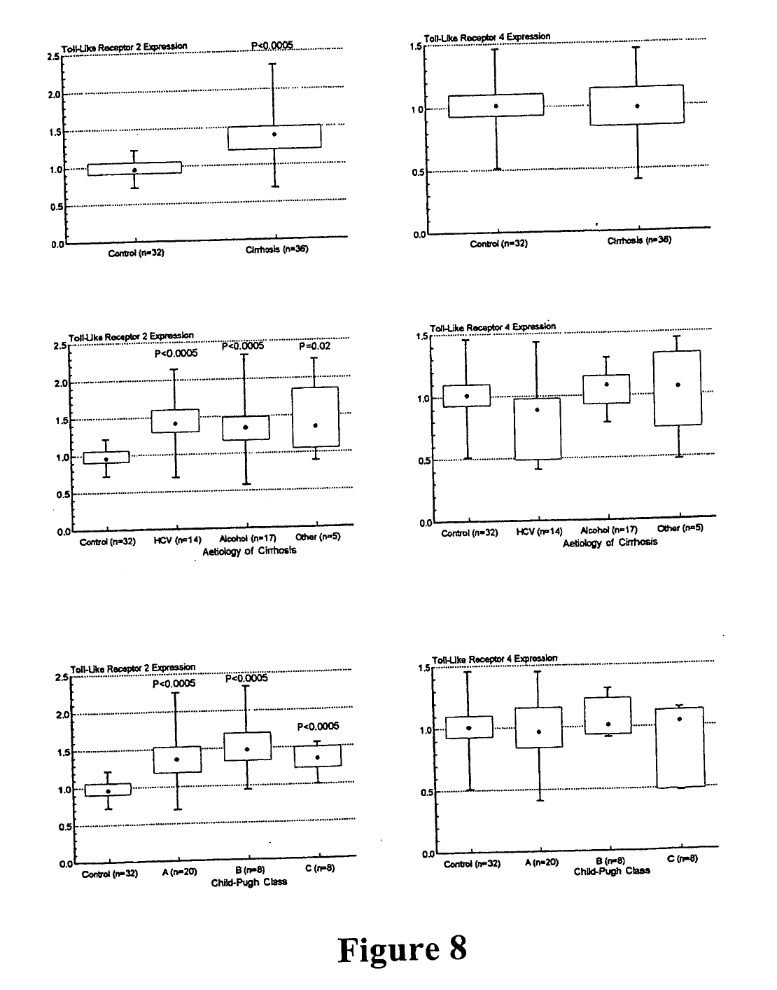

TLR-4 expression on CD14^{+ve} peripheral blood monocytes (FIG. 7) was not significantly different in cirrhotic patients and controls, even in Child-Pugh class A patients in whom significantly elevated plasma endotoxin levels were documented (FIG. 8). In contrast, TLR-2 expression (FIG. 7) was significantly increased in patients with cirrhosis. This up-regulation occurred irrespective of the aetiology of cirrhosis or Child-Pugh classification (FIG. 8) and was highly correlated with serum levels of TNF-α (r=0.49; P<0.0005), sTNF RI (r=0.53; P<0.0005) and sTNF RII (r=0.63; P<0.0005).

In Vitro Production of TNF-α by PBMCs

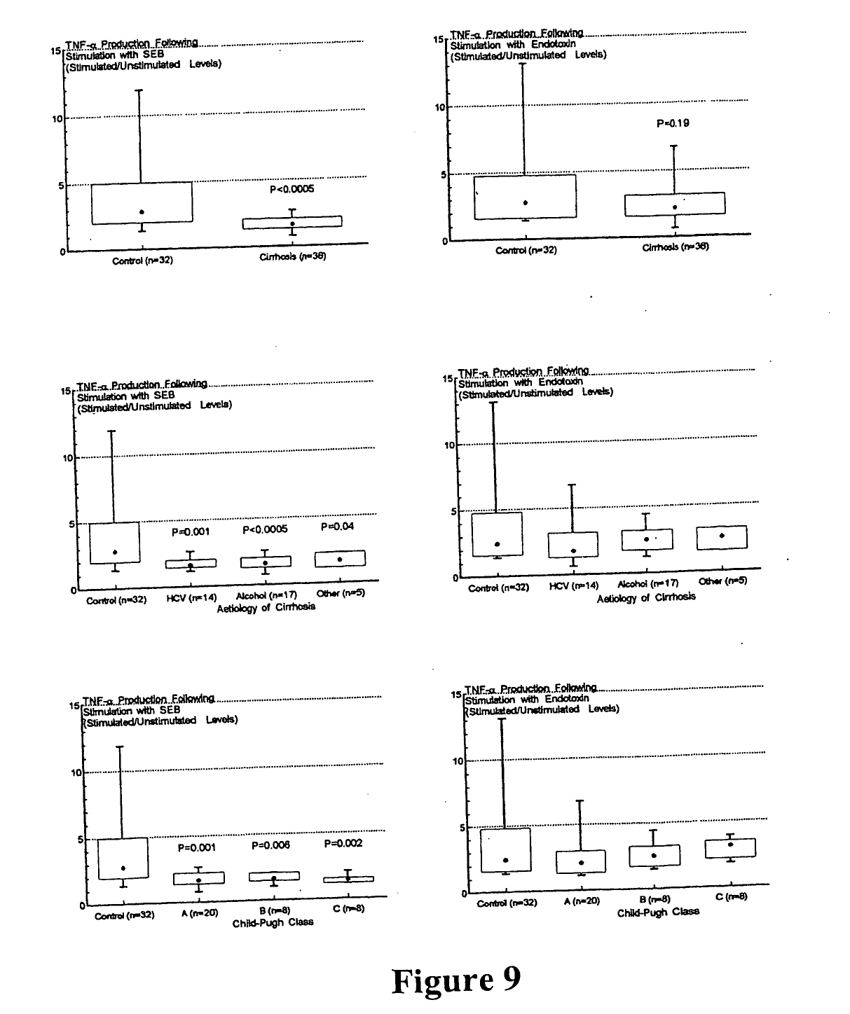

In vitro production of TNF-α following stimulation with endotoxin was not significantly different in cirrhotic patients and controls, even in Child-Pugh class A patients in whom increased plasma endotoxin levels had been documented (FIG. 9). In contrast, in vitro production of TNF-α following stimulation with SEB, although increased in comparison to unstimulated values, was increased to a significantly lesser extent than in control subjects, irrespective of aetiology of cirrhosis or Child-Pugh classification (FIG. 9).

A significant inverse correlation was found between in vitro production of TNF-α by PBMC's stimulated with SEB and serum TNF-α levels (r=−0.32; P=0.02).

Influence of Lactulose on Study Parameters

A greater number of Child-Pugh class B and C patients were receiving treatment with lactulose than their class A counterparts (7/16 {43.8%} versus 1/20 {5%}, respectively). This high prevalence of treatment with lactulose appeared to account for the finding that plasma endotoxin levels were not increased in the Child-Pugh class B and C groups overall. Plasma endotoxin values in the subgroup of Child-Pugh B and C patients treated with lactulose were not significantly different from those in control subjects; conversely, levels in Child-Pugh B and C patients not receiving lactulose were significantly higher than those found in either controls or lactulose-treated patients in this category (Table 6).

In contrast to endotoxin values, lactulose treatment did not significantly influence any other study parameter. Thus, serum concentrations of TNF-α and soluble TNF RI and sTNF RII along with PBMC expression of TLR-2 were each significantly increased and in vitro production of TNF-α by PBMCs in response to SEB was significantly reduced compared to control levels in both lactulose-treated and -non-treated groups. PBMC expression of TLR-4 and in vitro production of TNF-α by PBMCs in response to endotoxin did not differ significantly from control values in either lactulose-treated or -non-treated groups (Table 6).

Oral Supplementation of Cirrhotic Patients with the Synbiotic Gram-Positive Gut Flora Regimen

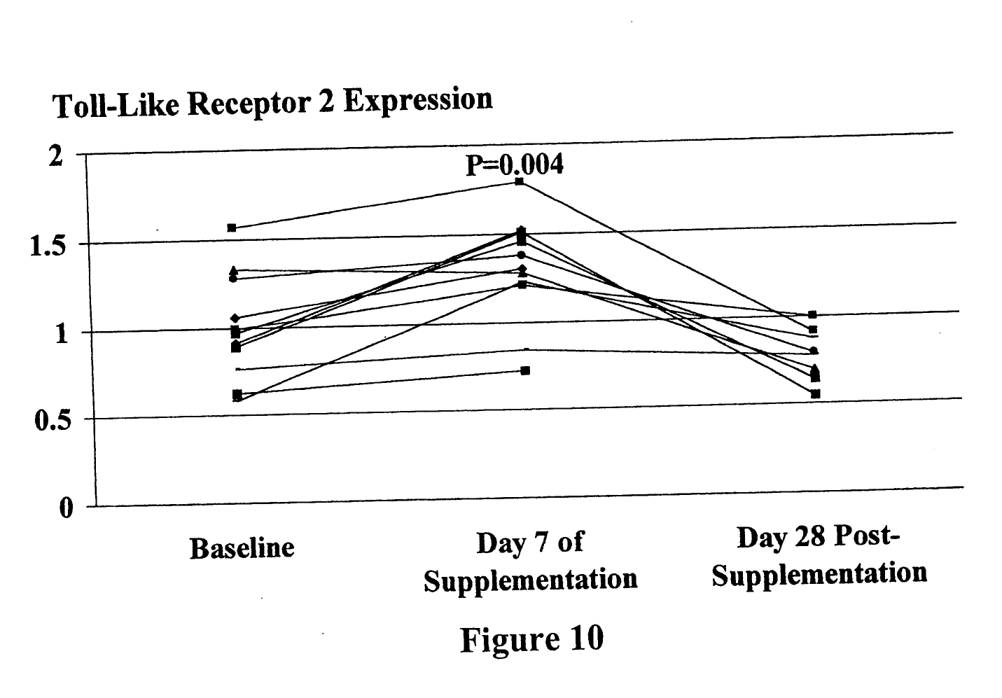

CD14^{+ve} PBMC expression of TLR-2 in cirrhotic patients was significantly increased after seven days of oral supplementation with the synbiotic Gram-positive gut flora regimen compared to pre-supplementation values. Levels fell substantially by day 28 post-cessation of supplementation in all eight patients in whom such follow-up data could be obtained (three patients developed exclusion criteria including gastrointestinal haemorrhage {n=1} and infection requiring use of antibiotics {n=2} prior to this time-point) (FIG. 10).

Serum TNF-α levels after seven days of supplementation were increased by a median 20% (range: 1-129%) in comparison to pre-supplementation levels in 8/11 (72.7%) cirrhotic patients. Levels fell towards baseline by day 28 post-cessation of supplementation in all six of these eight patients in whom follow-up data could be obtained.

In vitro TNF-α production by PBMCs in response to stimulation with SEB after seven days of supplementation was reduced by a median 46% (range: 8-67%) in comparison to pre-supplementation levels in 8/11 (72.7%) cirrhotic patients, including seven patients in whom supplementation was associated with an increase in serum TNF-α levels. Follow-up data at day 28 post-cessation of supplementation could be obtained in six of these eight patients. PBMC responsiveness to SEB improved towards baseline in three and persisted in the remaining three such patients.

Administration of the synbiotic supplement was well-tolerated without any reported adverse events or change in general clinical state.

### EXAMPLE 4

### In Vitro Analysis of HBV and TLR Expression

Hepatitis B Stimulation of Whole Blood

Peripheral blood was collected into Li-heparin tubes from 6 healthy volunteers. Whole blood was diluted with an equal volume of RPMI supplemented with antibiotics and 5% fetal bovine serum. 1 ml of diluted blood was stimulated with wildtype 1.5 (genotype A) HBV at a ratio of 1, 10 and 50 virus particles to 1 peripheral blood cell. Diluted blood was incubated at 37° C. with gentle rotation in tightly capped 5 ml polystyrene tubes. After 20 hours, culture supernatants were collected for TNF-alpha ELISA. The remaining cells were stained for flow cytometric analysis.

The HBV was prepared from transduction of cells using recombinant HBV/Baculovirus system as previously described (Delaney 4^{th} and Isom; Hepatology; 28:1134-46, 1998).

TNF-alpha ELISA

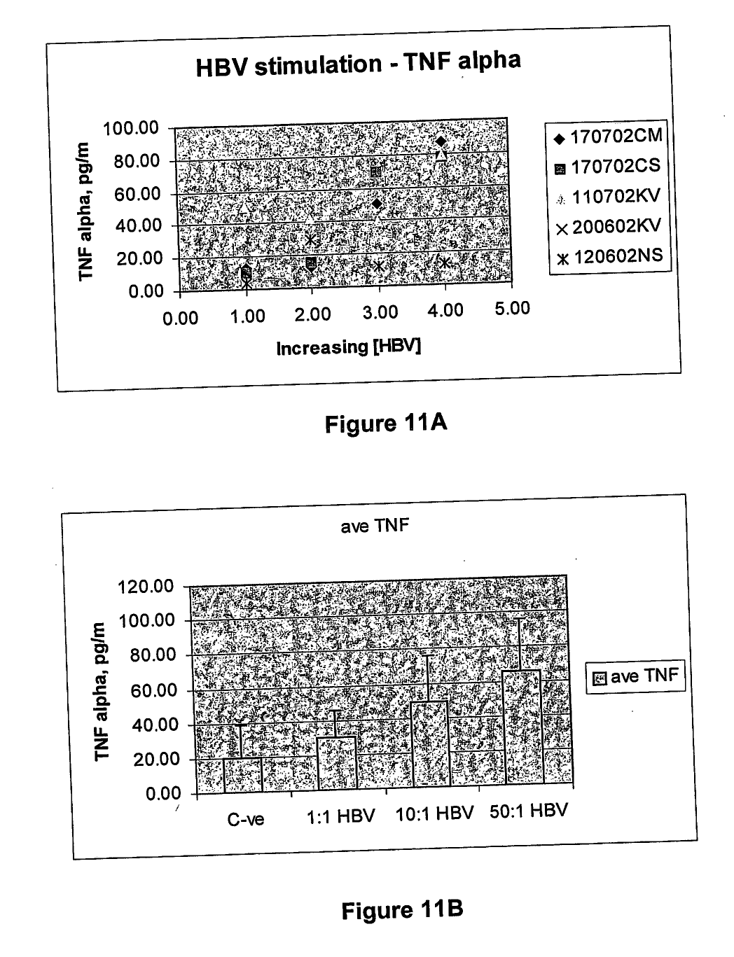

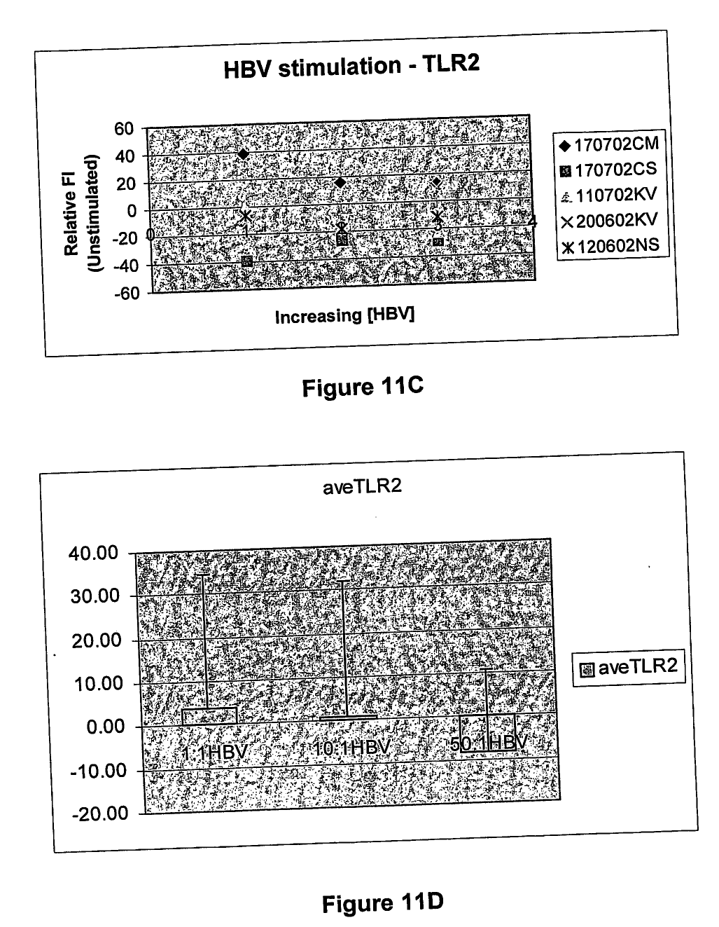

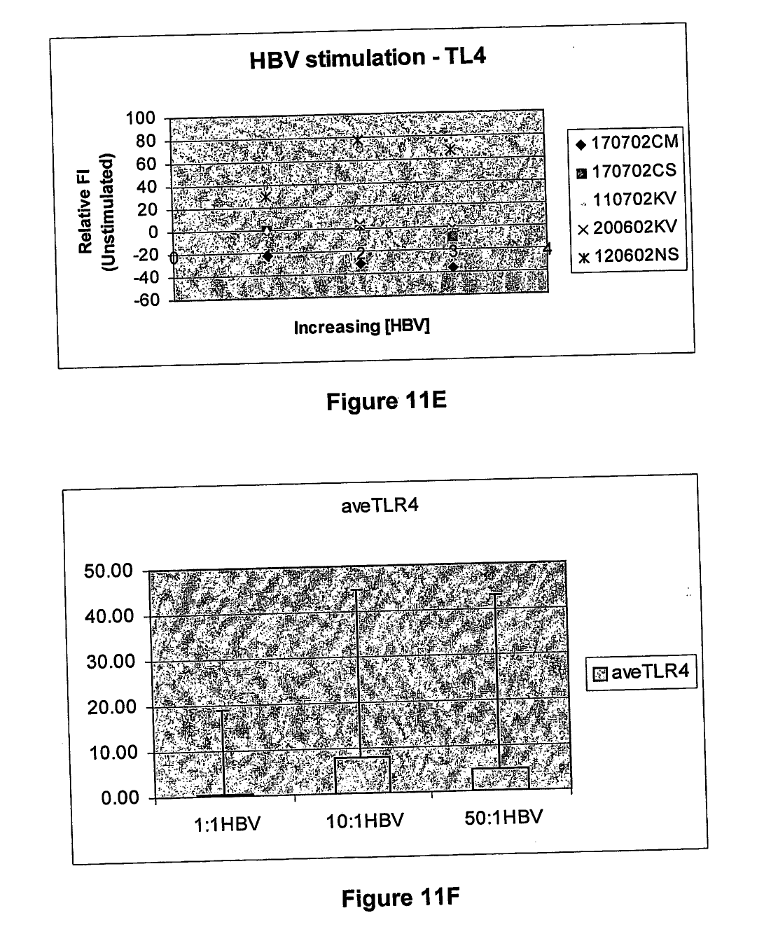

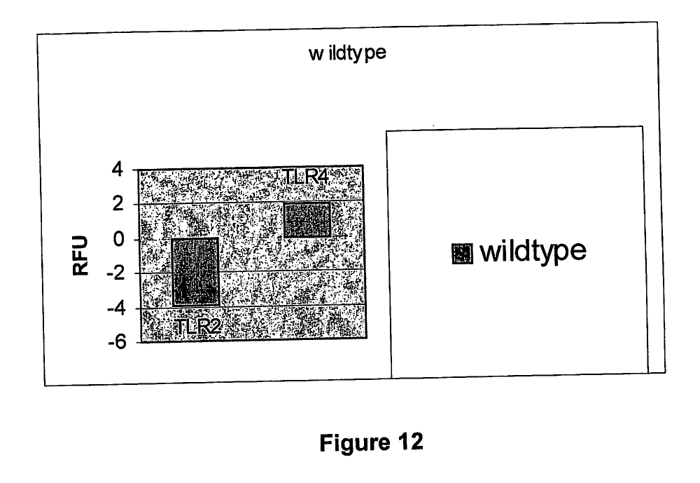

TNF-alpha from whole blood culture supernatants was measured by capture ELISA using TNF-alpha OptEIA set (BD) according to the manufacturer's specifications. Sensitivity was 8 pg/ml. The TNF alpha results for all patients in shown in FIG. 11a and b.

Flow Cytometry

Cell surface staining was performed on peripheral blood lymphocytes using CD14-PerCP (MφP9; BD), TLR2-FITC (TL2.1; eBioscience) and TLR4-PE (HTA125; eBioscience) antibodies. Appropriate isotype controls were used. Based on their scatter profile, monocytes were gated picking up the lymphocyte tail on a FACSCalibur flow cytometer (BD). A total of 8000 CD14 positive monocytes were acquired for each sample. Data was analysed using FlowJo software (Tree Star Inc.). Relative fluorescence intensity was determined by subtracting the geometric mean fluorescence intensity of the unstimulated control from the stimulated sample. The TLR2 and TLR4 results for all 6 patients following stimulation with wild type HBV is shown in FIGS. 11(c, d, e, and f)

HBV Baculovirus Infected HepG2 (Hershy)

The HBV was prepared from transduction of cells using recombinant HBV/Baculovirus system as previously described (Delaney 4^{th} and Isom; Hepatology. 1998, supra).

HepG2 (Hershy) cells were transdued with HBV 1:3 wildtype, or mock baculovirus infected and grown for 7 days prior to harvesting and staining for flow cytometry. Some cells were reserved for total RNA extraction using the RNeasy mini kit (Qiagen) following the Manufacturer's specifications.

Flow Cytometry

Cell surface staining was performed on HepG2 cells using TLR2-FITC (TL2.1; eBioscience) and TLR4-PE (HTA125; eBioscience) antibodies. Appropriate isotype controls were used. Dead cells were gated out based on their scatter profile, on a FACSCalibur flow cytometer (BD). A total of 10000 cells were acquired for each sample. Data was analysed using FlowJo software (Tree Star Inc.). Relative fluorescence intensity was determined by subtracting the geometric mean fluorescence intensity of the mock infected cells from the wildtype infected cells.

QPCR

Total RNA was reversed transcribed using random hexamers prior to real time PCR analysis of the cDNA. PCR was performed in triplicate using TaqMan Universal PCR Master Mix and Assays-On-Demand Gene Expression Assay probes and primers (Applied Biosystems) in a final 10 μl volume. Signal detection was via ABI Prism 7700 sequence detection system programmed to 50° C., 2 min; 95° C. 10 min; 40 cycles of 95° C., 15 sec; 60° C., 1 min. The threshold cycle (C_{T}), values of each gene were compared with the C_{T} value of 18S (ΔC_{T}) and relative expression units (REU) calculated for each sample. Hence, REU=2ˆC_{T}(gene of interest)−C_{T}(18S)=2ˆC_{T}

TABLE 5
Clinical details of patients with cirrhosis

| Child-Pugh | | Median Age | Gender | | | Portal Hypertension | Treatment with |
|---|---|---|---|---|---|---|---|
| Class | n | (Range) (years) | M/F | Aetiology of Cirrhosis | | (%) | Lactulose (%) |
| A | 20 | 50 (30-78) | 15/5 | Alcohol: | n = 7 | 12/20 (60.0) | 1/20 (5.0) |
| | | | | HCV: | n = 10 | | |
| | | | | HBV: | n = 2 | | |
| | | | | PBC: | n = 1 | | |
| B | 8 | 61 (49-74) | 6/2 | Alcohol: | n = 6 | 7/8 (87.5) | 2/8 (25.0) |
| | | | | HCV: | n = 2 | | |
| C | 8 | 45 (33-70) | 6/2 | Alcohol: | n = 4 | 8/8 (100) | 5/8 (62.5) |
| | | | | HCV: | n = 2 | | |
| | | | | HBV: | n = 1 | | |
| | | | | PSC: | n = 1 | | |

HCV: Hepatitis C virus
HBV: Hepatitis B virus
PBC: Primary biliary cirrhosis;
PSC: Primary sclerosing cholangitis

TABLE 6
Influence of lactulose treatment on study parameters

| | | | Child-Pugh B/C Patients | | | |
|---|---|---|---|---|---|---|
| | Controls (n = 32) | | No Lactulose (n = 9) | | Lactose-Treated (n = 7) | |
| | Median (Range) | | Median (Range) | | Median (Range) | |
| Plasma endotoxin (pg/mL) | 25 | (3-62) | 49 | (14-84) ^{a, b} | 13 | (5-49) |
| Serum TNF-α (pg/mL) | 1.7 | (0.8-3.4) | 3.1 | (1.8-14.1) ^{c} | 4.2 | (2.9-14.2) ^{d} |
| Serum soluble TNF receptor I (pg/mL) | 1643 | (446-3997) | 3852 | (1867-6687) ^{d} | 3878 | (1442-4571) ^{e} |
| Serum soluble TNF receptor II (pg/mL) | 2438 | (10-4885) | 5933 | (4321-5933) ^{d} | 5440 | (3046-5933) ^{f} |
| Expression of TLR-2 on CD14^{+ve} PBMCs | 0.96 | (0.72-1.28) | 1.48 | (1.24-2.32) ^{d} | 1.29 | (0.94-1.50) ^{g} |
| Expression of TLR-4 on CD14^{+ve} PBMCs | 1.00 | (0.51-1.48) | 0.98 | (0.50-1.32) | 1.06 | (0.45-1.13) |
| In vitro production of TNF-α by PBMCs in response to | 2.5 | (1.4-13.1) | 3.0 | (1.5-4.5) | 3.1 | (1.7-3.9) |
| endotoxin | | | | | | |
| In vitro production of TNF-α by PBMCs in response to SEB | 2.8 | (1.4-11.9) | 1.6 | (1.0-2.1) ^{d} | 1.5 | (1.2-2.1) ^{d} |

Bold text indicates significant difference compared to controls;
^{a} P = 0.05 compared to controls;
^{b} P = 0.02 compared to lactulose-treated group;
^{c} P = 0.0009 compared to controls;
^{d} P < 0.0005 compared to controls;
^{e} P = 0.02 compared to controls;
^{f} P = 0.001 compared to controls;
^{g} P = 0.007 compared to controls

### EXAMPLE 5

### Predicting Outcome of Therapeutic Intervention

Using the methods provided in Example 2, subjects potentially exposed to HBV are screened for their levels of TLR-2 and/or TLR-4 prior to therapeutic intervention. When a subject exhibits a change in the level of TLR-2 and/or TLR-4 during early phase treatment (i.e. a trend to normalization of levels of TLR-2 and/or TLR-4) then this predicts that the therapy is working. The change in levels may be an elevation or reduction compared to pre-treatment and/or levels of standardized normal controls.

A similar diagnostic or predictive process may be applied to subjects potentially exposed or infected with HCV.

Those skilled in the art will appreciate that the invention described herein is susceptible to variations and modifications other than those specifically described. It is to be understood that the invention includes all such variations and modifications. The invention also includes all of the steps, features, compositions and compounds referred to or indicated in this specification, individually or collectively, and any and all combinations of any two or more of said steps or features.

### BIBLIOGRAPHY

Adams et al., Cancer Res. 53: 4026-4034, 1993

Akashi et al., J Immunol. 164: 3471-3475, 2000

Akira et al., Nature Immunology 2: 675-680, 2001

Andus et al., Hepatology 13: 364-375, 1991

Baltimore, J. Virol. 54: 401-407, 1985

Bandyopadhyay and Temin, Mol. Cell. Biol. 4: 749-754, 1984

Berglund et al., Biotechnology 11: 916-920, 1993

Berkner et al., BioTechniques 6; 616-629, 1988

Berkner, Curr. Top. Microbiol. Immunol. 158: 39-66, 1992

Bieback et al., J Virol. 76: 8729-8736, 2002

Bigatello et al., Am J Gastroenterol. 82: 11-15, 1987

Breakefield and Geller, Mol. Neurobiol. 1: 339-371, 1987

Buchschacher and Panganiban, J. Virol. 66: 2731-2739, 1982

Chothia et al., J. Mol. Biol. 196: 901, 1987

Chothia et al., J. Mol. Biol. 227: 799, 1992

Coligan et al., “Current Protocols in Immunology, John Wiley & Sons, Inc., 1991-1997

Cumber et al., J. Immunol. 149: 120-126, 1992

Davies & Riechmann, FEBS Lett. 339: 285-290, 1994

Delaney 4^{th} and Isom; Hepatol., 28:1134-46, 1998.

Deviere et al., Gastroenterology 103: 1296-1301, 1992

Enomoto et al., Gastroenterology 115: 443-451, 1998

Enomoto et al., J Gastroenterol Hepatol. 15(Suppl): D20-D25, 2000

Erickson et al., Science 249: 527-533, 1990

Erlich, eds., PCR Technology, Stockton Press, NY, 1989

Fink et al., Ann. Rev. Neurosci. 19: 265-287, 1996

Fink et al., Hum. Gene Ther. 3: 11-19, 1992

Fodor et al., Science 251: 767-777, 1991

Freese et al., Biochem. Pharmacol. 40: 2189-2199, 1990

French J Biomed Sci. 8: 20-27, 2001

Fukui et al., J Hepatol. 12: 162-169, 1991

Gefter et al., Somatic Cell Genet. 3: 231-236, 1977

Genesca et al., Am J Gastroenterol. 94:169-177, 1999

Glockshuber et al., Biochem. 29: 1363-1367, 1990

Goeddel et al., Nucleic Acids Res. 8: 4057-1980

Gorziglia and Kapikian, J. Virol. 66: 4407-4412, 1992

Haeberle et al., J Infect Dis. 186:1199-1206, 2002

Hamers-Casterman et al., Nature 363: 446-448, 1993

Hanck et al., Gut 49: 106-111, 2001

Helseth et al., J. Virol. 64: 2416-2420, 1990

Hodgson, Bio/Technology 9: 19-21, 1991

Iimuro et al., Hepatology 26: 1530-1537, 1997

Johnson et al., “Peptide Turn Mimetics” in Biotechnology and Pharmacy, Pezzuto et al., Eds., Chapman and Hall, New York, 1993

Johnson et al., J. Virol. 66: 2952-2965, 1992

Jones et al., Nature 321: 522-525, 1986

Kabat et al., “Sequences of Proteins of Immunological Interest”, U.S. Department of Health and Human Sciences, 1983

Kazal et al., Nature Medicine 2: 753-759, 1996

Kennet et al., Monoclonal Antibodies and Hybridomas: A New Dimension in Biological Analyses, pp 376-384, Plenum Press, New York, 1980

Khoruts et al., Hepatology 13: 267-276, 1991

Kohler and Milstein, Eur. J Immunol. 6(7): 511-519, 1976

Köhler and Milstein, Nature 256: 495-499, 1975

Kostelny et al., J. Immunol. 148: 1547-1553, 1992

Kozbor et al., Methods in Enzymology 121: 140, 1986

Krebber et al., J. Immunol. Methods 201(1): 35-55, 1997

Ku & Schutz, Proc. Natl. Acad. Sci. USA 92: 6552-6556, 1995

Kurt-Jones et al., Nat Immunol. 1: 398-401, 2000

Lawn et al., Nucleic Acids Res. 2: 6103, 1981

Lee et al., Scand J Gastroenterol. 31: 500-505, 1996

Lewis et al., J. Pathol. 195: 66-71, 2001

Lin et al., J Hepatol. 22: 165-172, 1995

Chan et al., Scand J Gastroenterol. 32: 942-946, 1997

Liu et al., Proc. Natl. Acad. Sci. USA 84: 3439-3443, 1987

Mackay et al., Nucleic Acids Res 30(6): 1292-1305, 2002

Madzak et al., J. Gen. Virol. 73: 1533-1536, 1992

Manrekar et al., Alcohol Clin Exp Res. 21: 1226-1231, 1997

Margolskee, Curr. Top., Microbiol. Immunol. 158: 67-95, 1992;

Matsumoto et al., Biochem Biophys Res Commun. 293: 1364-1369, 2002

Medzhitov et al., N Engl J Med. 343: 338-344, 2000

Miller et al., J. Virol. 62: 4337-4345, 1988

Miller et al., Mol. Cell. Biol. 5: 431-437, 1985

Miller, Curr. Top. Microbiol. Immunol. 158: 1-24, 1992

Moss, Curr. Top. Microbiol. Immunol. 158: 25-38, 1992

Moss, Proc. Natl. Acad. Sci. USA 93: 11341-11348, 1996

Mullis et al., Quant. Biol. 51: 263, 1987

Muzyczka, Curr. Top. Microbiol. Immunol. 158: 97-129, 1992;

Naldini et al., Science 272: 263-267, 1996

Nanji et al., Am J Pathol. 142: 367-373, 1993

Neuman et al., J Gastroenterol Hepatol. 17: 196-202, 2002

O'Reilly et al., Biotechniques 25: 824-830, 1998

Ohi et al., Gene 89: 279-282, 1990;

Page et al., J. Virol. 64: 5270-5276, 1990

Petropoulos et al., J Viol. 66: 3391-3397, 1992

Plückthun et al., In Antibody engineering: A practical approach, 203-252, 1996

Plünckthun, Biochem 31: 1579-1584, 1992

Pugh et al., Br J Surg. 60: 646-649, 1973

Quantin et al., Proc. Natl. Acad. Sci. USA 89: 2581-2584, 1992

Reiter et al., Biochem. 33: 5451-5459, 1994

Reiter et al., Cancer Res. 54: 2714-2718, 1994

Reiter et al., J. Biol. Chem. 269:18327-18331, 1994

Remington's Pharmaceutical Sciences, 18^{th} Ed., 1990, Mack Publishing, Company, Easton, Pa., U.S.A.

Richmann et al., Nature 332: 323-327, 1988

Rivera et al., Am J Physiol. 275: G1252-1258, 1998

Rosenfeld et al., Cell 68: 143-155, 1992

Russell and Hirata, Nature Genetics 18: 323-328, 1998

Schafer et al., Z Gastroenterol. 33: 503-508, 1995

Schneider et al., Nature Genetics 18: 180-183, 1998

Shimada et al., J. Clin. Invest. 88: 1043-1047, 1991

Shulman et al., Nature 276: 269-270, 1978

Sorge et al., Mol. Cell. Biol. 4: 1730-1737, 1984

Stratford-Perricaudet et al., Hum. Gene Ther. 1: 241-256, 1990

Summerton and Weller, Antisense and Nucleic Acid Drug Development 7: 187-195, 1997

Takeuchi et al., Immunity 11: 443-451, 1999

Tapping et al., J Immunol. 165: 5780-5787, 2000

Tilg et al., Gastroenterology. 103: 264-274, 1992

Tilg et al., Hepatology 18: 1132-1138, 1993

Toyama et al., Monoclonal Antibody, Experiment Manual”, published by Kodansha Scientific, 1987

Trowbridge, J. Exp. Med. 148(1): 313-323, 1978

van Leeuwen et al., Surgery 110: 169-174, 1991

Verhoeyen et al., Science 239: 1534-1536, 1988

Volk et al., J. Virol. 42(1): 220-227, 1982

von Baehr et al., Gut 47: 281-287, 2000

Walker, J. Biochem. Mol. Toxicology 15(3): 121-127, 2001

Ward et al., Nature 341: 544-546, 1989

Webber et al., Mol. Immunol. 32: 249-258, 1995

Wells, Methods Enzymol. 202: 2699-2705, 1991

Wilkinson et al., Nucleic Acids Res. 20: 2233-2239, 1992

Winter and Milstein, Nature 349: 293, 1991

Yin et al., Gastroenterology 117: 942-952, 1999

Yoshimura et al., J Immunol. 163: 1-5, 1999

1-64. (canceled)

65. A method for detecting the presence of infection by a pathogenic agent, said method comprising determining the level of a cell surface marker selected from the group consisting of Toll-like receptors and homologs thereof wherein a change in said level is indicative of infection by said pathogenic agent.

66. The method of claim 65, wherein said Toll-Like receptor is selected from the group consisting of TLR-2, TLR-4 or a homolog thereof.

67. The method of claim 65, wherein said marker is affected in a manner selected from the group consisting of up-regulated and down-regulated.

68. The method of claim 65, wherein said marker is determined by analyzing the mRNA or protein associated with said marker.

69. The method of claim 65, wherein said pathogenic agent is selected from the group consisting of Salmonella, Escherichia, Klebsiella, Pasteurella, Bacillus, Clostridium, Corynebacterium, Mycoplasma, Ureaplasma, Actinomyces, Mycobacterium, Chlamydia, Chlamydophila, Leptospira, Spirochaeta, Borrelia, Treponema, Pseudomonas, Burkholderia, Dichelobacter, Haemophilus, Ralstonia, Xanthomonas, Moraxella, Acinetobacter, Branhamella, Kingella, Erwinia, Enterobacter, Arozona, Citrobacter, Proteus, Providencia, Yersinia, Shigella, Edwardsiella, Vibrio, Rickettsia, Coxiella, Ehrlichia, Arcobacteria, Peptostreptococcus, Candida, Aspergillus, Trichomonas, Bacterioides, Coccidiomyces, Pneumocystis, Cryptosporidium, Porphyromonas, Actinobacillus, Lactococcus, Lactobacillua, Zymononas, Saccharomyces, Propionibacterium, Streptomyces, Penicillum, Neisseria, Staphylococcus, Campylobacter, Streptococcus, Enterococcus, Helicobacter, human immunodeficiency virus (HIV), Varicella-Zoster virus (VZV), herpes simplex virus (HSV), human papillomavirus (HPV), Hepatitis B virus (HBV), Hepatitis A virus (HAV), rhinovirus, echovirus, Coxsackievirus, cytomegalovirus, flavivirus, Ebola virus, paramyxovirus, influenza virus, enterovirus, Epstein-Barr virus, Marburg virus, polio virus, rabies virus, rubella virus, smallpox virus, rubeola virus, vaccina virus, adenovirus, rotavirus Hepatitis C virus (HCV) and Hepatitis B virus (HBV).

70. A method for detecting a disease condition, said method comprising determining the level of a cell surface marker selected from the group consisting of Toll-like receptors and homologs thereof wherein a change in said level is indicative of said disease condition.

71. The method of claim 70, wherein said Toll-Like receptor is selected from the group consisting of TLR-2, TLR-4 or a homolog thereof.

72. The method of claim 70, wherein said marker is affected in a manner selected from the group consisting of up-regulated and down-regulated.

73. The method of claim 70, wherein said marker is determined by analyzing the mRNA or protein associated with said marker.

74. The method of claim 70, wherein said pathogenic agent is selected from the group consisting of Salmonella, Escherichia, Klebsiella, Pasteurella, Bacillus, Clostridium, Corynebacterium, Mycoplasma, Ureaplasma, Actinomyces, Mycobacterium, Chlamydia, Chlamydophila, Leptospira, Spirochaeta, Borrelia, Treponema, Pseudomonas, Burkholderia, Dichelobacter, Haemophilus, Ralstonia, Xanthomonas, Moraxella, Acinetobacter, Branhamella, Kingella, Erwinia, Enterobacter, Arozona, Citrobacter, Proteus, Providencia, Yersinia, Shigella, Edwardsiella, Vibrio, Rickettsia, Coxiella, Ehrlichia, Arcobacteria, Peptostreptococcus, Candida, Aspergillus, Trichomonas, Bacterioides, Coccidiomyces, Pneumocystis, Cryptosporidium, Porphyromonas, Actinobacillus, Lactococcus, Lactobacillua, Zymononas, Saccharomyces, Propionibacterium, Streptomyces, Penicillum, Neisseria, Staphylococcus, Campylobacter, Streptococcus, Enterococcus, Helicobacter, human immunodeficiency virus (HIV), Varicella-Zoster virus (VZV), herpes simplex virus (HSV), human papillomavirus (HPV), Hepatitis B virus (HBV), Hepatitis A virus (HAV), rhinovirus, echovirus, Coxsackievirus, cytomegalovirus, flavivirus, Ebola virus, paramyxovirus, influenza virus, enterovirus, Epstein-Barr virus, Marburg virus, polio virus, rabies virus, rubella virus, smallpox virus, rubeola virus, vaccina virus, adenovirus, rotavirus Hepatitis C virus (HCV) and Hepatitis B virus (HBV).

75. A method of detecting a predisposition to infection by a pathogenic agent, said method comprising determining the level of a cell surface marker selected from the group consisting of Toll-like receptors and homologs thereof wherein a change in said level is indicative of said predisposition to infection by a pathogenic agent.

76. The method of claim 75, wherein said level is compared to a sample selected from the group consisting of a pre-treatment sample and a control sample

77. The method of claim 75, wherein said Toll-Like receptor is selected from the group consisting of TLR-2, TLR-4 or a homolog thereof.

78. The method of claim 75, wherein said marker is affected in a manner selected from the group consisting of up-regulated and down-regulated.

79. The method of claim 75, wherein said marker is determined by analyzing the mRNA or protein associated with said marker.

80. The method of claim 75, wherein said pathogenic agent is selected from the group consisting of Salmonella, Escherichia, Klebsiella, Pasteurella, Bacillus, Clostridium, Corynebacterium, Mycoplasma, Ureaplasma, Actinomyces, Mycobacterium, Chlamydia, Chlamydophila, Leptospira, Spirochaeta, Borrelia, Treponema, Pseudomonas, Burkholderia, Dichelobacter, Haemophilus, Ralstonia, Xanthomonas, Moraxella, Acinetobacter, Branhamella, Kingella, Erwinia, Enterobacter, Arozona, Citrobacter, Proteus, Providencia, Yersinia, Shigella, Edwardsiella, Vibrio, Rickettsia, Coxiella, Ehrlichia, Arcobacteria, Peptostreptococcus, Candida, Aspergillus, Trichomonas, Bacterioides, Coccidiomyces, Pneumocystis, Cryptosporidium, Porphyromonas, Actinobacillus, Lactococcus, Lactobacillua, Zymononas, Saccharomyces, Propionibacterium, Streptomyces, Penicillum, Neisseria, Staphylococcus, Campylobacter, Streptococcus, Enterococcus, Helicobacter, human immunodeficiency virus (HIV), Varicella-Zoster virus (VZV), herpes simplex virus (HSV), human papillomavirus (HPV), Hepatitis B virus (HBV), Hepatitis A virus (HAV), rhinovirus, echovirus, Coxsackievirus, cytomegalovirus, flavivirus, Ebola virus, paramyxovirus, influenza virus, enterovirus, Epstein-Barr virus, Marburg virus, polio virus, rabies virus, rubella virus, smallpox virus, rubeola virus, vaccina virus, adenovirus, rotavirus Hepatitis C virus (HCV) and Hepatitis B virus (HBV).

81. A method for monitoring a response to a therapeutic protocol to prevent infection by a pathogenic agent, said method comprising determining the level of a cell surface marker selected from the group consisting of Toll-like receptors and homologs thereof wherein the efficacy of said therapeutic response is determined by a change in said level.

82. The method of claim 81, wherein said level is compared to a sample selected from the group consisting of a pre-treatment sample and a control sample

83. The method of claim 81, wherein said Toll-Like receptor is selected from the group consisting of TLR-2, TLR-4 or a homolog thereof.

84. The method of claim 81, wherein said marker is affected in a manner selected from the group consisting of up-regulated and down-regulated.

85. The method of claim 81, wherein said marker is determined by analyzing the mRNA or protein associated with said marker.

86. The method of claim 81, wherein said pathogenic agent is selected from the group consisting of Salmonella, Escherichia, Klebsiella, Pasteurella, Bacillus, Clostridium, Corynebacterium, Mycoplasma, Ureaplasma, Actinomyces, Mycobacterium, Chlamydia, Chlamydophila, Leptospira, Spirochaeta, Borrelia, Treponema, Pseudomonas, Burkholderia, Dichelobacter, Haemophilus, Ralstonia, Xanthomonas, Moraxella, Acinetobacter, Branhamella, Kingella, Erwinia, Enterobacter, Arozona, Citrobacter, Proteus, Providencia, Yersinia, Shigella, Edwardsiella, Vibrio, Rickettsia, Coxiella, Ehrlichia, Arcobacteria, Peptostreptococcus, Candida, Aspergillus, Trichomonas, Bacterioides, Coccidiomyces, Pneumocystis, Cryptosporidium, Porphyromonas, Actinobacillus, Lactococcus, Lactobacillua, Zymononas, Saccharomyces, Propionibacterium, Streptomyces, Penicillum, Neisseria, Staphylococcus, Campylobacter, Streptococcus, Enterococcus, Helicobacter, human immunodeficiency virus (HIV), Varicella-Zoster virus (VZV), herpes simplex virus (HSV), human papillomavirus (HPV), Hepatitis B virus (HBV), Hepatitis A virus (HAV), rhinovirus, echovirus, Coxsackievirus, cytomegalovirus, flavivirus, Ebola virus, paramyxovirus, influenza virus, enterovirus, Epstein-Barr virus, Marburg virus, polio virus, rabies virus, rubella virus, smallpox virus, rubeola virus, vaccina virus, adenovirus, rotavirus Hepatitis C virus (HCV) and Hepatitis B virus (HBV).

87. A method for monitoring a response to a therapeutic protocol to prevent development of a disease condition, said method comprising determining the level of a cell surface marker selected from the group consisting of Toll-like receptors and homologs thereof wherein the efficacy of said therapeutic response is determined by a change in said level.

88. The method of claim 87, wherein said level is compared to a sample selected from the group consisting of a pre-treatment sample and a control sample.

89. The method of claim 87, wherein said Toll-Like receptor is selected from the group consisting of TLR-2, TLR-4 or a homolog thereof.

90. The method of claim 87, wherein said marker is affected in a manner selected from the group consisting of up-regulated and down-regulated.

91. The method of claim 87, wherein said marker is determined by analyzing the mRNA or protein associated with said marker.

92. The method of claim 87, wherein said pathogenic agent is selected from the group consisting of Salmonella, Escherichia, Klebsiella, Pasteurella, Bacillus, Clostridium, Corynebacterium, Mycoplasma, Ureaplasma, Actinomyces, Mycobacterium, Chlamydia, Chlamydophila, Leptospira, Spirochaeta, Borrelia, Treponema, Pseudomonas, Burkholderia, Dichelobacter, Haemophilus, Ralstonia, Xanthomonas, Moraxella, Acinetobacter, Branhamella, Kingella, Erwinia, Enterobacter, Arozona, Citrobacter, Proteus, Providencia, Yersinia, Shigella, Edwardsiella, Vibrio, Rickettsia, Coxiella, Ehrlichia, Arcobacteria, Peptostreptococcus, Candida, Aspergillus, Trichomonas, Bacterioides, Coccidiomyces, Pneumocystis, Cryptosporidium, Porphyromonas, Actinobacillus, Lactococcus, Lactobacillua, Zymononas, Saccharomyces, Propionibacterium, Streptomyces, Penicillum, Neisseria, Staphylococcus, Campylobacter, Streptococcus, Enterococcus, Helicobacter, human immunodeficiency virus (HIV), Varicella-Zoster virus (VZV), herpes simplex virus (HSV), human papillomavirus (HPV), Hepatitis B virus (HBV), Hepatitis A virus (HAV), rhinovirus, echovirus, Coxsackievirus, cytomegalovirus, flavivirus, Ebola virus, paramyxovirus, influenza virus, enterovirus, Epstein-Barr virus, Marburg virus, polio virus, rabies virus, rubella virus, smallpox virus, rubeola virus, vaccina virus, adenovirus, rotavirus Hepatitis C virus (HCV) and Hepatitis B virus (HBV).

93. A method for determining whether a subject will respond to therapeutic intervention of infection by a pathogenic agent, said method comprising determining the level of a cell surface marker selected from the group consisting of Toll-like receptors and homologs thereof wherein the efficacy of said therapeutic intervention is determined by a change in said level.

94. The method of claim 93, wherein said level is compared to a sample selected from the group consisting of a pre-treatment sample and a control sample

95. The method of claim 93, wherein said Toll-Like receptor is selected from the group consisting of TLR-2, TLR-4 or a homolog thereof.

96. The method of claim 93, wherein said marker is affected in a manner selected from the group consisting of up-regulated and down-regulated.

97. The method of claim 93, wherein said marker is determined by analyzing the mRNA or protein associated with said marker.

98. The method of claim 93, wherein said pathogenic agent is selected from the group consisting of Salmonella, Escherichia, Klebsiella, Pasteurella, Bacillus, Clostridium, Corynebacterium, Mycoplasma, Ureaplasma, Actinomyces, Mycobacterium, Chlamydia, Chlamydophila, Leptospira, Spirochaeta, Borrelia, Treponema, Pseudomonas, Burkholderia, Dichelobacter, Haemophilus, Ralstonia, Xanthomonas, Moraxella, Acinetobacter, Branhamella, Kingella, Erwinia, Enterobacter, Arozona, Citrobacter, Proteus, Providencia, Yersinia, Shigella, Edwardsiella, Vibrio, Rickettsia, Coxiella, Ehrlichia, Arcobacteria, Peptostreptococcus, Candida, Aspergillus, Trichomonas, Bacterioides, Coccidiomyces, Pneumocystis, Cryptosporidium, Porphyromonas, Actinobacillus, Lactococcus, Lactobacillua, Zymononas, Saccharomyces, Propionibacterium, Streptomyces, Penicillum, Neisseria, Staphylococcus, Campylobacter, Streptococcus, Enterococcus, Helicobacter, human immunodeficiency virus (HIV), Varicella-Zoster virus (VZV), herpes simplex virus (HSV), human papillomavirus (HPV), Hepatitis B virus (HBV), Hepatitis A virus (HAV), rhinovirus, echovirus, Coxsackievirus, cytomegalovirus, flavivirus, Ebola virus, paramyxovirus, influenza virus, enterovirus, Epstein-Barr virus, Marburg virus, polio virus, rabies virus, rubella virus, smallpox virus, rubeola virus, vaccina virus, adenovirus, rotavirus Hepatitis C virus (HCV) and Hepatitis B virus (HBV).

99. A method for determining whether a subject will respond to prophylactic intervention of infection by a pathogenic agent, said method comprising determining the level of a cell surface marker selected from the group consisting of Toll-like receptors and homologs thereof wherein the efficacy of said prophylactic intervention is determined by a change in said level.

100. The method of claim 99, wherein said level is compared to a sample selected from the group consisting of a pre-treatment sample and a control sample

101. The method of claim 99, wherein said Toll-Like receptor is selected from the group consisting of TLR-2, TLR-4 or a homolog thereof.

102. The method of claim 99, wherein said marker is affected in a manner selected from the group consisting of up-regulated and down-regulated.

103. The method of claim 99, wherein said marker is determined by analyzing the mRNA or protein associated with said marker.

104. The method of claim 99, wherein said pathogenic agent is selected from the group consisting of Salmonella, Escherichia, Klebsiella, Pasteurella, Bacillus, Clostridium, Corynebacterium, Mycoplasma, Ureaplasma, Actinomyces, Mycobacterium, Chlamydia, Chlamydophila, Leptospira, Spirochaeta, Borrelia,Treponema, Pseudomonas, Burkholderia, Dichelobacter, Haemophilus, Ralstonia, Xanthomonas, Moraxella, Acinetobacter, Branhamella, Kingella, Erwinia, Enterobacter, Arozona, Citrobacter, Proteus, Providencia, Yersinia, Shigella, Edwardsiella, Vibrio, Rickettsia, Coxiella, Ehrlichia, Arcobacteria, Peptostreptococcus, Candida, Aspergillus, Trichomonas, Bacterioides, Coccidiomyces, Pneumocystis, Cryptosporidium, Porphyromonas, Actinobacillus, Lactococcus, Lactobacillua, Zymononas, Saccharomyces, Propionibacterium, Streptomyces, Penicillum, Neisseria, Staphylococcus, Campylobacter, Streptococcus, Enterococcus, Helicobacter, human immunodeficiency virus (HIV), Varicella-Zoster virus (VZV), herpes simplex virus (HSV), human papillomavirus (HPV), Hepatitis B virus (HBV), Hepatitis A virus (HAV), rhinovirus, echovirus, Coxsackievirus, cytomegalovirus, flavivirus, Ebola virus, paramyxovirus, influenza virus, enterovirus, Epstein-Barr virus, Marburg virus, polio virus, rabies virus, rubella virus, smallpox virus, rubeola virus, vaccina virus, adenovirus, rotavirus Hepatitis C virus (HCV) and Hepatitis B virus (HBV).

105. A method for predicting the outcome of a therapeutic protocol to prevent infection by a pathogenic agent, said method comprising determining the level of a cell surface marker selected from the group consisting of Toll-like receptors and homologs thereof wherein the efficacy of said therapeutic protocol is determined by a change in said level.

106. The method of claim 105, wherein said level is compared to a sample selected from the group consisting of a pre-treatment sample and a control sample

107. The method of claim 105, wherein said Toll-Like receptor is selected from the group consisting of TLR-2, TLR-4 or a homolog thereof.

108. The method of claim 105, wherein said marker is affected in a manner selected from the group consisting of up-regulated and down-regulated.

109. The method of claim 105, wherein said marker is determined by analyzing the mRNA or protein associated with said marker.

110. The method of claim 105, wherein said pathogenic agent is selected from the group consisting of Salmonella, Escherichia, Klebsiella, Pasteurella, Bacillus, Clostridium, Corynebacterium, Mycoplasma, Ureaplasma, Actinomyces, Mycobacterium, Chlamydia, Chlamydophila, Leptospira, Spirochaeta, Borrelia, Treponema, Pseudomonas, Burkholderia, Dichelobacter, Haemophilus, Ralstonia, Xanthomonas, Moraxella, Acinetobacter, Branhamella, Kingella, Erwinia, Enterobacter, Arozona, Citrobacter, Proteus, Providencia, Yersinia, Shigella, Edwardsiella, Vibrio, Rickettsia, Coxiella, Ehrlichia, Arcobacteria, Peptostreptococcus, Candida, Aspergillus, Trichomonas, Bacterioides, Coccidiomyces, Pneumocystis, Cryptosporidium, Porphyromonas, Actinobacillus, Lactococcus, Lactobacillua, Zymononas, Saccharomyces, Propionibacterium, Streptomyces, Penicillum, Neisseria, Staphylococcus, Campylobacter, Streptococcus, Enterococcus, Helicobacter, human immunodeficiency virus (HIV), Varicella-Zoster virus (VZV), herpes simplex virus (HSV), human papillomavirus (HPV), Hepatitis B virus (HBV), Hepatitis A virus (HAV), rhinovirus, echovirus, Coxsackievirus, cytomegalovirus, flavivirus, Ebola virus, paramyxovirus, influenza virus, enterovirus, Epstein-Barr virus, Marburg virus, polio virus, rabies virus, rubella virus, smallpox virus, rubeola virus, vaccina virus, adenovirus, rotavirus Hepatitis C virus (HCV) and Hepatitis B virus (HBV).

111. A method for predicting the outcome of a therapeutic protocol to prevent development of a disease condition, said method comprising determining the level of a cell surface marker selected from the group consisting of Toll-like receptors and homologs thereof wherein the efficacy of said therapeutic protocol is determined by a change in said level.

112. The method of claim 111, wherein said level is compared to a sample selected from the group consisting of a pre-treatment sample and a control sample.

113. The method of claim 111, wherein said Toll-Like receptor is selected from the group consisting of TLR-2, TLR-4 or a homolog thereof.

114. The method of claim 111, wherein said marker is affected in a manner selected from the group consisting of up-regulated and down-regulated.

115. The method of claim 111, wherein said marker is determined by analyzing the mRNA or protein associated with said marker.

116. The method of claim 111, wherein said pathogenic agent is selected from the group consisting of Salmonella, Escherichia, Klebsiella, Pasteurella, Bacillus, Clostridium, Corynebacterium, Mycoplasma, Ureaplasma, Actinomyces, Mycobacterium, Chlamydia, Chlamydophila, Leptospira, Spirochaeta, Borrelia, Treponema, Pseudomonas, Burkholderia, Dichelobacter, Haemophilus, Ralstonia, Xanthomonas, Moraxella, Acinetobacter, Branhamella, Kingella, Erwinia, Enterobacter, Arozona, Citrobacter, Proteus, Providencia, Yersinia, Shigella, Edwardsiella, Vibrio, Rickettsia, Coxiella, Ehrlichia, Arcobacteria, Peptostreptococcus, Candida, Aspergillus, Trichomonas, Bacterioides, Coccidiomyces, Pneumocystis, Cryptosporidium, Porphyromonas, Actinobacillus, Lactococcus, Lactobacillua, Zymononas, Saccharomyces, Propionibacterium, Streptomyces, Penicillum, Neisseria, Staphylococcus, Campylobacter, Streptococcus, Enterococcus, Helicobacter, human immunodeficiency virus (HIV), Varicella-Zoster virus (VZV), herpes simplex virus (HSV), human papillomavirus (HPV), Hepatitis B virus (HBV), Hepatitis A virus (HAV), rhinovirus, echovirus, Coxsackievirus, cytomegalovirus, flavivirus, Ebola virus, paramyxovirus, influenza virus, enterovirus, Epstein-Barr virus, Marburg virus, polio virus, rabies virus, rubella virus, smallpox virus, rubeola virus, vaccina virus, adenovirus, rotavirus Hepatitis C virus (HCV) and Hepatitis B virus (HBV).

117. A method of treating a subject infected with a pathogenic agent, said method comprising administering to said subject an effective amount of an agent which affects the level of a cell surface marker selected from the group consisting of Toll-like receptors and homologs thereof.

118. The method of claim 117, wherein said Toll-Like receptor is selected from the group consisting of TLR-2, TLR-4 or a homolog thereof.

119. The method of claim 117, wherein said marker is affected in a manner selected from the group consisting of up-regulated and down-regulated.

120. The method of claim 117, wherein said marker is determined by analyzing the mRNA or protein associated with said marker.

121. The method of claim 117, wherein said pathogenic agent is selected from the group consisting of Salmonella, Escherichia, Klebsiella, Pasteurella, Bacillus, Clostridium, Corynebacterium, Mycoplasma, Ureaplasma, Actinomyces, Mycobacterium, Chlamydia, Chlamydophila, Leptospira, Spirochaeta, Borrelia, Treponema, Pseudomonas, Burkholderia, Dichelobacter, Haemophilus, Ralstonia, Xanthomonas, Moraxella, Acinetobacter, Branhamella, Kingella, Erwinia, Enterobacter, Arozona, Citrobacter, Proteus, Providencia, Yersinia, Shigella, Edwardsiella, Vibrio, Rickettsia, Coxiella, Ehrlichia, Arcobacteria, Peptostreptococcus, Candida, Aspergillus, Trichomonas, Bacterioides, Coccidiomyces, Pneumocystis, Cryptosporidium, Porphyromonas, Actinobacillus, Lactococcus, Lactobacillua, Zymononas, Saccharomyces, Propionibacterium, Streptomyces, Penicillum, Neisseria, Staphylococcus, Campylobacter, Streptococcus, Enterococcus, Helicobacter, human immunodeficiency virus (HIV), Varicella-Zoster virus (VZV), herpes simplex virus (HSV), human papillomavirus (HPV), Hepatitis B virus (HBV), Hepatitis A virus (HAV), rhinovirus, echovirus, Coxsackievirus, cytomegalovirus, flavivirus, Ebola virus, paramyxovirus, influenza virus, enterovirus, Epstein-Barr virus, Marburg virus, polio virus, rabies virus, rubella virus, smallpox virus, rubeola virus, vaccina virus, adenovirus, rotavirus Hepatitis C virus (HCV) and Hepatitis B virus (HBV).

122. The method of claim 117, wherein said agent is selected from the group consisting of a large chemical molecule, a small chemical molecule, a nucleic acid molecule, a peptide, a polypeptide, a protein, a RNAi, an antisense molecule and an antibody.

123. A method of treating a subject having a disease condition, said method comprising administering to said subject an effective amount of an agent which affects the level of a cell surface marker selected from the group consisting of Toll-like receptors and homologs thereof.

124. The method of claim 123, wherein said Toll-Like receptor is selected from the group consisting of TLR-2, TLR-4 or a homolog thereof.

125. The method of claim 123, wherein said marker is affected in a manner selected from the group consisting of up-regulated and down-regulated.

126. The method of claim 123, wherein said marker is determined by analyzing the mRNA or protein associated with said marker.

127. The method of claim 123, wherein said pathogenic agent is selected from the group consisting of Salmonella, Escherichia, Klebsiella, Pasteurella, Bacillus, Clostridium, Corynebacterium, Mycoplasma, Ureaplasma, Actinomyces, Mycobacterium, Chlamydia, Chlamydophila, Leptospira, Spirochaeta, Borrelia, Treponema, Pseudomonas, Burkholderia, Dichelobacter, Haemophilus, Ralstonia, Xanthomonas, Moraxella, Acinetobacter, Branhamella, Kingella, Erwinia, Enterobacter, Arozona, Citrobacter, Proteus, Providencia, Yersinia, Shigella, Edwardsiella, Vibrio, Rickettsia, Coxiella, Ehrlichia, Arcobacteria, Peptostreptococcus, Candida, Aspergillus, Trichomonas, Bacterioides, Coccidiomyces, Pneumocystis, Cryptosporidium, Porphyromonas, Actinobacillus, Lactococcus, Lactobacillua, Zymononas, Saccharomyces, Propionibacterium, Streptomyces, Penicillum, Neisseria, Staphylococcus, Campylobacter, Streptococcus, Enterococcus, Helicobacter, human immunodeficiency virus (HIV), Varicella-Zoster virus (VZV), herpes simplex virus (HSV), human papillomavirus (HPV), Hepatitis B virus (HBV), Hepatitis A virus (HAV), rhinovirus, echovirus, Coxsackievirus, cytomegalovirus, flavivirus, Ebola virus, paramyxovirus, influenza virus, enterovirus, Epstein-Barr virus, Marburg virus, polio virus, rabies virus, rubella virus, smallpox virus, rubeola virus, vaccina virus, adenovirus, rotavirus Hepatitis C virus (HCV) and Hepatitis B virus (HBV).

128. The method of claim 123, wherein said agent is selected from the group consisting of a large chemical molecule, a small chemical molecule, a nucleic acid molecule, a peptide, a polypeptide, a protein, a RNAi, an antisense molecule and an antibody.

129. A method of treating a subject having a predisposition to infection with a pathogenic agent, said method comprising administering to said subject an effective amount of an agent which affects the level of a cell surface marker selected from the group consisting of Toll-like receptors and homologs thereof.

130. The method of claim 129, wherein said Toll-Like receptor is selected from the group consisting of TLR-2, TLR-4 or a homolog thereof.

131. The method of claim 129, wherein said marker is affected in a manner selected from the group consisting of up-regulated and down-regulated.

132. The method of claim 129, wherein said marker is determined by analyzing the mRNA or protein associated with said marker.

133. The method of claim 129, wherein said pathogenic agent is selected from the group consisting of Salmonella, Escherichia, Klebsiella, Pasteurella, Bacillus, Clostridium, Corynebacterium, Mycoplasma, Ureaplasma, Actinomyces, Mycobacterium, Chlamydia, Chlamydophila, Leptospira, Spirochaeta, Borrelia, Treponema, Pseudomonas, Burkholderia, Dichelobacter, Haemophilus, Ralstonia, Xanthomonas, Moraxella, Acinetobacter, Branhamella, Kingella, Erwinia, Enterobacter, Arozona, Citrobacter, Proteus, Providencia, Yersinia, Shigella, Edwardsiella, Vibrio, Rickettsia, Coxiella, Ehrlichia, Arcobacteria, Peptostreptococcus, Candida, Aspergillus, Trichomonas, Bacterioides, Coccidiomyces, Pneumocystis, Cryptosporidium, Porphyromonas, Actinobacillus, Lactococcus, Lactobacillua, Zymononas, Saccharomyces, Propionibacterium, Streptomyces, Penicillum, Neisseria, Staphylococcus, Campylobacter, Streptococcus, Enterococcus, Helicobacter, human immunodeficiency virus (HIV), Varicella-Zoster virus (VZV), herpes simplex virus (HSV), human papillomavirus (HPV), Hepatitis B virus (HBV), Hepatitis A virus (HAV), rhinovirus, echovirus, Coxsackievirus, cytomegalovirus, flavivirus, Ebola virus, paramyxovirus, influenza virus, enterovirus, Epstein-Barr virus, Marburg virus, polio virus, rabies virus, rubella virus, smallpox virus, rubeola virus, vaccina virus, adenovirus, rotavirus Hepatitis C virus (HCV) and Hepatitis B virus (HBV).

134. The method of claim 129, wherein said agent is selected from the group consisting of a large chemical molecule, a small chemical molecule, a nucleic acid molecule, a peptide, a polypeptide, a protein, a RNAi, an antisense molecule and an antibody.

135. A composition comprising a compound selected from the group consisting of Toll-like receptors, antagonists of Toll-like receptors, agonists of Toll-like receptors and homologs of Toll-like receptors and one or more pharmaceutically acceptable carriers, and/or diluents.

136. The method of claim 135, wherein said Toll-Like receptor is selected from the group consisting of TLR-2, TLR-4 or a homolog thereof.
